SEQUENCE LISTING (65 sequences)

SEQ ID NO: 1 - DNA, 3966 bp, Glycine max
caccacgtatccctgaaaagagagaaaaacacactaagccaaagccaaagcagcaatggttaaagacacaaagcctttagcctatgctgctaataatggataccaaaaggaagcttttgatcccagtgctcctccaccgtttaagattgcagaaatcagagttgcaataccaaaacattgctgggtcaagaatccatggagatccctcagttatgttctcagggatgtgcttgtaattgctgcattgatggctgctgcaagtcacttcaacaactggcttctctggctaatctattggcccattcaaggaacaatgttctgggctctgtttgttcttggacatgattggtaattaattaatttgttgttacttttttgttataatatgaatctcacacactgctttgttatgcctacctcatttcatttggctttagacaacttaaatttgagatctttattatgttttttgcttatatggtaaagtgattcattcttcacattgaattgaacagtggccatggaagcttttcagacagcccttttctaaatagcctggtgggacacatcttgcattcctcaattcttgtgccataccatggatggttagttcatcccggcttttttgtttgtcattggaagttcttttattgattcaatttttatagcgtgttcggaaacgcgtttcagaaaataatgaaatacatcttgaatctgaaagttataacttttagcttcattgtcattgaaagttcttttattaattatatttttattgcgtgtttggaatcccatttgagaaataagaaatcacgtttaaaatgtgaaagttataactattaacttttgactaaacttgaaaaaatcacatttttgatgtggaaccaaatctgatttgagaaccaagttgattttgatggattttgcaggagaattagccacagaactcaccatcaaaatcatggacacattgagaaggatgaatcctgggttccagtatgtgattaactacttcctctatagttatttttgattcaattaaatttatttatttaataagttcaagaaaaaaggaatctttatacttcagataaagctgttcttgaacatttttttttgtcattatcttagttaaccgagaagatttacaagaatctagacaacatgacaagacttgttagattcactgtgccatttccattgtttgtgtatccaatttatttggtgagtgcttttttttttttacttggaagactacaacacattattattattataatatggttcaaatcaatgacttttaatttctttgtgatgtgcactccattttcagttctcaagaagccccggaaaggaaggttctcacttcaatccctacagcaatctgttcccacccagtgagagaaagggaatagcaatatcaacactgtgttgggttaccatgttttctatgcttatctatctctccttcataactagtccagttctattgctcaagctctatggaattccatattgggtaattaaattactcttacattactttttcctctttttttttatgggtcttaactagtatcacaaaaatattggttaaaaaattttaaaaaaatatttattatgtaaatcataaaagaacataaaaaaaatgatgaataacataattttcgtctcttattaaaaatatttttattttaaatttcttaatcaatatatttagaatctggttaacattttttgaatatttcaattctccaattaaaaatttgaaatagtcaccattaattatgtaattgtttgaacacgtgcagatatttgttatgtggctggactttgtcacatacttgcatcaccatggtcatcatcagaaactgccttggtatcgcggcaaggtaacaaaaataaatagaaaatagtgagtgaacacttaaatgttagatactaccttcttcttcttttttttttttttttgaggttaatgctagataatagctagaaagagaaagaaagacaaatataggtaaaaataaataatataacctgggaagaagaaaacataaaaaaagaaataatagagtctacgtaatgtttggatttttgagtgaaatggtgttcacctaccattactcaaagattctgttgtctacgtagtgtttggactttggagtgaaatggtgttcacctaccattactcagattctgttgtgtcccttagttactgtcttatattcttagggtatattctttattttacatccttttcacatcttacttgaaaagatttttaattattcattgaaatattaacgtgacagttaaattaaaataataaaaaattcgttaaaacttcaaataaataagagtgaaaggatcatcatttttcttctttcttttattgcgttattaatcatgcttctcttcttttttttcttcgctttccacccatatcaaattcatgtgaagtatgagaaaatcacgattcaatggaaagctacaggaactttttttgttttgtttttataatcggaattaatttatactccattttttcacaataaatgttacttagtgccttaaagataatatttgaaaaattaaaaaaattattaatacactgtactactatataatatttgacatatatttaacatgattttctattgaaaatttgtatttattattttttaatcaaaacccataaggcattaatttacaagacccatttttcatttatagctttacctgtgatcatttatagctttaagggacttagatgttacaatcttaattacaagtaaatatttatgaaaaacatgtgtcttaccccttaaccttacctcaacaaagaaagtgtgataagtggcaacacacgtgttgcttttttggcccagcaataacacgtgtttttgtggtgtacaaaaatggacaggaatggagttatttaagaggtggtctcacaactgtggatcgtgactatggttggatcaataacattcaccatgacattggcacccatgttattcaccatcttttccctcaaattcctcattatcacctcgttgaagcggtatattttactattattactcacctaaaaagaatgcaattagtacatttgttttatctcttggaagttagtcattttcagttgcatgattgtaatgttctctctatttttaaaccatgttttcacacctacttcgtttaaaataagaatgtggatactattctaatttctattaacttcttttaaaaaataatgtaaaactagtattaaaaaagaggaaatagattacactctactaatactaatagtataaaaaaaattacattgttattttatcacaaataattatatataattaatttttacaatcattatcttaaaagtcatgtatgatatacagtttttacatgctttggtacttattgtaaagttagtgatttattcattatttatgttatataattggcataaatatcatgtaaccagctcactatactataatgggaacttggtggtgaaaggggtttacaaccctcttttctaggtgtaggtgctttgatacttctggtccctttttatatcaatataaattatattttgctgataaaaaaaacattattaatatataatcattaacttctttaaaaaccgtacctaaaactttatattattaaaaagaagattgagatcagcaaaagaaaaaaaaattaacagtcatttgaattcactgcagacacaagcagcaaaatcagttcttggagagtattaccgtgagccagaaagatctgcaccattaccatttcatctaataaagtatttaattcagagtatgagacaagaccacttcgtaagtgacactggagatgtggtttattatcagactgattctctgcaccttcactcgcaccgagactgagtttcaatttttgggttatttattggattctagctactcaaat

SEQ ID NO: 2 - DNA, 4013 bp, Glycine max
caccacgtatccctgagaaaagagaggaaccacactctctaagccaaagcaaaagcagcagcagcagcaatggttaaagacacaaagcctttagcctatgctgccaataatggataccaacaaaagggttcttcttttgattttgatcctagcgctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattggtaataatttttgtgtttcttactcttttttttttttttttgtttatgatatgaatctcacacattgttctgttatgtcatttcttcttcatttggctttagacaacttaaatttgagatctttattatgtttttgcttatatggtaaagtgattcttcattatttcattcttcattgattgaattgaacagtggccatggaagcttttcagatagccctttgctgaatagcctggtgggacacatcttgcattcctcaattcttgtgccataccatggatggttagttcatactggcttttttgtttgttcatttgtcattgaaaaaaaatcttttgttgattcaattatttttatagtgtgtttggaagcccgtttgagaaaataagaaatcgcatctggaatgtgaaagttataactatttagcttcatctgtcgttgcaagttcttttattggttaaatttttatagcgtgctaggaaacccattcgagaaaataagaaatcacatctggaatgtgaaagttataactgttagcttctgagtaaacgtggaaaaaccacattttggatttggaaccaaattttatttgataaatgacaaccaaattgattttgatggattttgcaggagaattagccacagaactcaccatcaaaaccatggacacattgagaaggatgagtcatgggttccagtatgtgattaattgcttctcctatagttgttcttgattcaattacattttatttatttggtaggtccaagaaaaaagggaatctttatgcttcctgaggctgttcttgaacatggctcttttttatgtgtcattatcttagttaacagagaagatttacaagaatctagacagcatgacaagactcattagattcactgtgccatttccattgtttgtgtatccaatttatttggtgagtgattttttgacttggaagacaacaacacattattattataatatggttcaaaacaatgactttttctttatgatgtgaactccattttttagttttcaagaagccccggaaaggaaggctctcacttcaatccctacagcaatctgtttccacccagtgagagaaaaggaatagcaatatcaacactgtgttgggctaccatgttttctctgcttatctatctctcattcataactagtccacttctagtgctcaagctctatggaattccatattgggtaactaaattactcctacattgttactttttcctccttttttttattatttcaattctccaattggaaatttgaaatagttaccataattatgtaattgtttgatcatgtgcagatatttgttatgtggctggactttgtcacatacttgcatcaccatggtcaccaccagaaactgccttggtaccgcggcaaggtaacaaaaataaatagaaaatagtgggtgaacacttaaatgcgagatagtaatacctaaaaaaagaaaaaaatataggtataataaataatataactttcaaaataaaaagaaatcatagagtctagcgtagtgtttggagtgaaatgatgttcacctaccattactcaaagattttgttgtgtcccttagttcattcttattattttacatatcttacttgaaaagactttttaattattcattgagatcttaaagtgactgttaaattaaaataaaaaacaagtttgttaaaacttcaaataaataagagtgaagggagtgtcatttgtcttctttcttttattgcgttattaatcacgtttctcttctctttttttttttttcttctctgctttccacccattatcaagttcatgtgaagcagtggcggatctatgtaaatgagtggggggcaattgcacccacaagattttattttttatttgtacaggaataataaaataaaactttgcccccataaaaaataaatattttttcttaaaataatgcaaaataaatataagaaataaaaagagaataaattattattaattttattattttgtactttttatttagtttttttagcggttagatttttttttcatgacattatgtaatcttttaaaagcatgtaatatttttattttgtgaaaataaatataaatgatcatattagtctcagaatgtataaactaataataattttatcactaaaagaaattctaatttagtccataaataagtaaaacaagtgacaattatattttatatttacttaatgtgaaataatacttgaacattataataaaacttaatgacaggagatattacatagtgccataaagatattttaaaaaataaaatcattaatacactgtactactatataatattcgatatatatttttaacatgattctcaatagaaaaattgtattgattatattttattagacatgaatttacaagccccgtttttcatttatagctcttacctgtgatctattgttttgcttcgctgtttttgttggtcaagggacttagatgtcacaatattaatactagaagtaaatatttatgaaaacatgtaccttacctcaacaaagaaagtgtggtaagtggcaacacacgtgttgcatttttggcccagcaataacacgtgtttttgtggtgtactaaaatggacaggaatggagttatttaagaggtggcctcaccactgtggatcgtgactatggttggatcaataacattcaccatgacattggcacccatgttatccaccatcttttcccccaaattcctcattatcacctcgttgaagcggtacattttattgcttattcacctaaaaacaatacaattagtacatttgttttatctcttggaagttagtcattttcagttgcatgattctaatgctctctccattcttaaatcatgttttcacacccacttcatttaaaataagaacgtgggtgttattttaatttctattcactaacatgagaaattaacttatttcaagtaataattttaaaatatttttatgctattattttattacaaataattatgtatattaagtttattgattttataataattatattaaaattatatcgatattaatttttgattcactgatagtgttttatattgttagtactgtgcatttattttaaaattggcataaataatatatgtaaccagctcactatactatactgggagcttggtggtgaaaggggttcccaaccctcctttctaggtgtacatgctttgatacttctggtaccttcttatatcaatataaattatattttgctgataaaaaaacatggttaaccattaaattctttttttaaaaaaaaaactgtatctaaactttgtattattaaaaagaagtctgagattaacaataaactaacactcatttgaattcactgcagacacaagcagcaaaaccagttcttggagattactaccgtgagccagaaagatctgcgccattaccatttcatctaataaagtatttaattcagagtatgagacaagaccacttcgtaagtgacactggagatgttgtttattatcagactgattctctgctcctccactcgcaacgagactgagtttcaaactttttgggttattatttattggattctagctactcaaat

SEQ ID NO: 3 - DNA, 2534 bp, Glycine max
atggttcaagcacagcctctacaacatgttggtaatggtgcagggaaagaagatcaagcttattttgatccaagtgctccaccacccttcaagattgcaaatatcagagcagcaattccaaaacattgctgggagaagaacacattgagatctctgagttatgttctgagggatgtgttggtagtgactgcattggtagctgcagcaatcggcttcaatagctggttcttctggccactctattggcctgcacaaggcacaatgttttgggcactttttgttcttggacatgattggtaactaattattattacaaattgttatgttatgttatgttatgttgttgtgcctttttctcagtgatgctttagtcatttcatttcacttggttatgcatgattgttcgttcatatgttctgtcatggtgagttctaatttgattgatgcatggaacagtggtcatggaagtttttcaaacagtcctttgttgaacagcattgtgggccacatcttgcactcttcaattcttgtaccataccatggatggtcggttccttttagcaacttttcatgttcactttgtccttaaatttttttttatgtttgttaaaaaatctttggtctgatttaacaacctaaccatttttacaactcatggattttttgcaggagaattagccacaggactcaccatcagaaccatggccatgttgagaaggatgaatcatgggttccggtattactatgagtttgcttgattaatttccacattttttctttcttcttaattttaatcagtggttagatttggttgtgttccaatagaagaaaagggggtatctagagagatgtgaatttcatgaagtggttcatgattatgtgtctttatgcctttatgtcagcttacagagaaagtttacaagaatctagacaacatgacaagaatgatgagattcactcttcctttccccatctttgcataccccttttatttggtgagaccctctttttccagaatgacagcattattttactatatagtacctcaatttttatatttctaaaattttgaattcttgaaattgaaaggaaaggactttattgggtctagcatctcactctctctttgtgatatgaaccatatatttcagtggagcagaagccctggaaaagaaggctctcatttcaacccttacagcaacttgttctctcctggtgagagaagagatgtgctaacttcaactctgtgttggggcatcatgctttctgtgcttctctatctttccctcacaatgggtccactttttatgctcaagctctatggggttccctatttggtaatctcactctcacactttctttatacatcgcacaccagtgtgggttatttgcaacctacaccgaagtaatgccctataattaatggggttaacacatgtccaagtccaatattttgttcacttatttgaacttgaacatgtgtagatcttcgtcatgtggctggatttcgtcacgtacttgcatcatcatggttacaagcagaaactgccttggtaccgtggccaggtatcccatttaacacaatttgtttcattaacattttaagagaatttttttttcaaaatagttttcgaaattaagcaaataccaagcaaattgttagatctacgcttgtacttgttttaaagtcaaattcatgaccaaattgtcctcacaagtccaaaccgtccactattttattttcacctactttatagcccaatttgtcatttggttacttcagaaaagagaaccccatttgtagtaaatatattatttatgaattatggtagtttcaacataaaacatatttatgtgcagttttgccatccttcaaaagaagatagaaacttactccatgttactctgtctatatgtaatttcacaggaatggagttatctaaggggtggtcttacaacagtagatcgcgactatggttggatcaacaacattcaccatgacattggcacccatgttatccatcaccttttccctcaaattccacattatcatttaatcgaagcggtattaattctctatttcacaagaaattattgtatgtctgcctatgtgatctaagtcaattttcacataacacatgatcaaactttcttaattctttcttctaaattgaaaaagtggattatatgtcaattgaaaattggtcaagaccacaaacatgtgatgatctcccaccttacatataataatttctcctattctacaatcaataatccttctatggtcctgaattgttcctttcttttttcattttcttattctttttgttgtcccacaatagactaaagcagcaaaggcagtgctaggaaagtattatcgtgagcctcagaaatctgggccattgccacttcatctaataaagtacttgctccacagcataagtcaggatcacttcgttagcgactctggcgacattgtgtactaccagactgattcccagctccacaaagattcttggacccagtccaactaa

SEQ ID NO: 4 - DNA, 17 bp, Artificial Sequence
  source: /note=“Description of Artificial Sequence Synthetic oligonucleotide”
tgggacacatcttgcat

SEQ ID NO: 5 - DNA, 17 bp, Artificial Sequence
  source: /note=“Description of Artificial Sequence Synthetic oligonucleotide”
ttgtgccataccatgga

SEQ ID NO: 6 - DNA, 17 bp, Artificial Sequence
  source: /note=“Description of Artificial Sequence Synthetic oligonucleotide”
tgggccacatcttgcac

SEQ ID NO: 7 - DNA, 17 bp, Artificial Sequence
  source: /note=“Description of Artificial Sequence Synthetic oligonucleotide”
ttgtaccataccatgga

SEQ ID NO: 8 - DNA, 17 bp, Artificial Sequence
  source: /note=“Description of Artificial Sequence Synthetic oligonucleotide”
ttgctgggtcaagaatc

SEQ ID NO: 9 - DNA, 17 bp, Artificial Sequence
  source: /note=“Description of Artificial Sequence Synthetic oligonucleotide”
agttatgttctcaggga

SEQ ID NO: 10 - DNA, 17 bp, Artificial Sequence
  source: /note=“Description of Artificial Sequence Synthetic oligonucleotide”
ttgctgggagaagaaca

SEQ ID NO: 11 - DNA, 17 bp, Artificial Sequence
  source: /note=“Description of Artificial Sequence Synthetic oligonucleotide”
agttatgttctgaggga

SEQ ID NO: 12 - DNA, 17 bp, Artificial Sequence
  source: /note=“Description of Artificial Sequence Synthetic oligonucleotide”
ttggcccattcaaggaa

SEQ ID NO: 13 - DNA, 17 bp, Artificial Sequence
  source: /note=“Description of Artificial Sequence Synthetic oligonucleotide”
tttgttcttggacatga

SEQ ID NO: 14 - DNA, 17 bp, Artificial Sequence
  source: /note=“Description of Artificial Sequence Synthetic oligonucleotide”
ttgccccattcaaggca

SEQ ID NO: 15 - DNA, 17 bp, Artificial Sequence
  source: /note=“Description of Artificial Sequence Synthetic oligonucleotide”
ttggcctgcacaaggca

SEQ ID NO: 16 - DNA, 17 bp, Artificial Sequence
  source: /note=“Description of Artificial Sequence Synthetic oligonucleotide”
ttagccacagaactcac

SEQ ID NO: 17 - DNA, 17 bp, Artificial Sequence
  source: /note=“Description of Artificial Sequence Synthetic oligonucleotide”
cacattgagaaggatga

SEQ ID NO: 18 - DNA, 17 bp, Artificial Sequence
  source: /note=“Description of Artificial Sequence Synthetic oligonucleotide”
ttagccacaggactcac

SEQ ID NO: 19 - DNA, 17 bp, Artificial Sequence
  source: /note=“Description of Artificial Sequence Synthetic oligonucleotide”
catgttgagaaggatga

SEQ ID NO: 20 - DNA, 1131 bp, Glycine max
atggttaaagacacaaagcctttagcctatgctgctaataatggataccaaaaggaagcttttgatcccagtgctcctccaccgtttaagattgcagaaatcagagttgcaataccaaaacattgctgggtcaagaatccatggagatccctcagttatgttctcagggatgtgcttgtaattgctgcattgatggctgctgcaagtcacttcaacaactggcttctctggctaatctattggcccattcaaggaacaatgttctgggctctgtttgttcttggacatgattgtggccatggaagcttttcagacagcccttttctaaatagcctggtgggacacatcttgcattcctcaattcttgtgccataccatggatggagaattagccacagaactcaccatcaaaatcatggacacattgagaaggatgaatcctgggttccattaaccgagaagatttacaagaatctagacaacatgacaagacttgttagattcactgtgccatttccattgtttgtgtatccaatttatttgttctcaagaagccccggaaaggaaggttctcacttcaatccctacagcaatctgttcccacccagtgagagaaagggaatagcaatatcaacactgtgttgggttaccatgttttctatgcttatctatctctccttcataactagtccagttctattgctcaagctctatggaattccatattggatatttgttatgtggctggactttgtcacatacttgcatcaccatggtcatcatcagaaactgccttggtatcgcggcaaggaatggagttatttaagaggtggtctcacaactgtggatcgtgactatggttggatcaataacattcaccatgacattggcacccatgttattcaccatcttttccctcaaattcctcattatcacctcgttgaagcgacacaagcagcaaaatcagttcttggagagtattaccgtgagccagaaagatctgcaccattaccatttcatctaataaagtatttaattcagagtatgagacaagaccacttcgtaagtgacactggagatgtggtttattatcagactgattctctgcaccttcactcgcaccgagactga

SEQ ID NO: 21 - DNA, 1143 bp, Glycine max
atggttaaagacacaaagcctttagcctatgctgccaataatggataccaacaaaagggttcttcttttgattttgatcctagcgctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgtttccacccagtgagagaaaaggaatagcaatatcaacactgtgttgggctaccatgttttctctgcttatctatctctcattcataactagtccacttctagtgctcaagctctatggaattccatattggatatttgttatgtggctggactttgtcacatacttgcatcaccatggtcaccaccagaaactgccttggtaccgcggcaaggaatggagttatttaagaggtggcctcaccactgtggatcgtgactatggttggatcaataacattcaccatgacattggcacccatgttatccaccatcttttcccccaaattcctcattatcacctcgttgaagcgacacaagcagcaaaaccagttcttggagattactaccgtgagccagaaagatctgcgccattaccatttcatctaataaagtatttaattcagagtatgagacaagaccacttcgtaagtgacactggagatgttgtttattatcagactgattctctgctcctccactcgcaacgagactga

SEQ ID NO: 22 - DNA, 1143 bp, Glycine max
atggttcaagcacagcctctacaacatgttggtaatggtgcagggaaagaagatcaagcttattttgatccaagtgctccaccacccttcaagattgcaaatatcagagcagcaattccaaaacattgctgggagaagaacacattgagatctctgagttatgttctgagggatgtgttggtagtgactgcattggtagctgcagcaatcggcttcaatagctggttcttctggccactctattggcctgcacaaggcacaatgttttgggcactttttgttcttggacatgattgtggtcatggaagtttttcaaacagtcctttgttgaacagcattgtgggccacatcttgcactcttcaattcttgtaccataccatggatggagaattagccacaggactcaccatcagaaccatggccatgttgagaaggatgaatcatgggttccgcttacagagaaagtttacaagaatctagacaacatgacaagaatgatgagattcactcttcctttccccatctttgcataccccttttatttgtggagcagaagccctggaaaagaaggctctcatttcaacccttacagcaacttgttctctcctggtgagagaagagatgtgctaacttcaactctgtgttggggcatcatgctttctgtgcttctctatctttccctcacaatgggtccactttttatgctcaagctctatggggttccctatttgatcttcgtcatgtggctggatttcgtcacgtacttgcatcatcatggttacaagcagaaactgccttggtaccgtggccaggaatggagttatctaaggggtggtcttacaacagtagatcgcgactatggttggatcaacaacattcaccatgacattggcacccatgttatccatcaccttttccctcaaattccacattatcatttaatcgaagcgactaaagcagcaaaggcagtgctaggaaagtattatcgtgagcctcagaaatctgggccattgccacttcatctaataaagtacttgctccacagcataagtcaggatcacttcgttagcgactctggcgacattgtgtactaccagactgattcccagctccacaaagattcttggacccagtccaactaa

SEQ ID NO: 23 - DNA, 1873 bp, Glycine max
ccatatactaatatttgcttgtattgatagcccctccgttcccaagagtataaaactgcatcgaataatacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtcatgatttcactctctctaatctctccattcattttgtagttgtcattatctttagatttttcactacctggtttaaaattgagggattgtagttctgttggtacatattacacattcagcaaaacaactgaaactcaactgaacttgtttatactttgacacagggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattgatggagcaaccaatgggccatagtgggagttatggaagttttgtcatgtattagtacataattagtagaatgttataaataagtggatttgccgcgtaatgactttgtgtgtattgtgaaacagcttgttgcgatcatggttataatgtaaaaataattctggtattaattacatgtggaaagtgttctgcttatagctttctgcct

SEQ ID NO: 24 - DNA, 3401 bp, Glycine max
ttttttctctgttgaaattgaaagtgttttccgggaaataaataaaataaattaaaatcttacacactctaggtaggtacttctaatttaatccacactttgactctatatatgttttaaaaataattataatgcgtacttactttctcattatactaaatttaacatcgatgattttattttctgtttctcttctttccacctacatacatcccaaaatttagggtgcaattttaagtttattaacacatgtttttagctgcatgctgcctttgtgtgtgctcaccaaattgcattcttctctttatatgttgtatttgaattttcacaccatatgtaaacaagattacgtacgtgtccatgatcaaatacaaatgctgtcttatactggcaatttgataaacagccgtccattttttctttttctctttaactatatatgctctagaatctctgaagattcctctgccatcgaatttctttcttggtaacaacgtcgtcgttatgttattattttattctatttttattttatcatatatatttcttattttgttcgaagtatgtcatattttgatcgtgacaattagattgtcatgtaggagtaggaatatcactttaaaacattgattagtctgtaggcaatattgtcttctttttcctcctttattaatatattttgtcgaagttttaccacaaggttgattcgctttttttgtccctttctcttgttctttttacctcaggtattttagtctttcatggattataagatcactgagaagtgtatgcatgtaatactaagcaccatagctgttctgcttgaatttatttgtgtgtaaattgtaatgtttcagcgttggctttccctgtagctgctacaatggtactgtatatctattttttgcattgttttcattttttcttttacttaatcttcattgctttgaaattaataaaacaatataatatagtttgaactttgaactattgcctattcatgtaattaacttattcactgactcttattgtttttctggtagaattcattttaaattgaaggataaattaagaggcaatacttgtaaattgacctgtcataattacacaggaccctgttttgtgcctttttgtctctgtctttggttttgcatgttagcctcacacagatatttagtagttgttctgcatacaagcctcacacgtatactaaaccagtggacctcaaagtcatggccttacacctattgcatgcgagtctgtgacacaacccctggtttccatattgcaatgtgctacgccgtcgtccttgtttgtttccatatgtatattgataccatcaaattattatatcatttatatggtctggaccattacgtgtactctttatgacatgtaattgagttttttaattaaaaaaatcaatgaaatttaactacgtagcatcatatagagataattgactagaaatttgatgacttattctttcctaatcatattttcttgtattgatagccccgctgtcccttttaaactcccgagagagtataaaactgcatcgaatattacaagatgcactcttgtcaaatgaagggggggaaatgatactacaagccactaggcatggtatgatgctaaattaaattgtgcctgcaccccaggatatttcatgtgggattcatcatttattgaggaaaactctccaaattgaatcgtgcatttatatttttttttccatttctagatttcttgaaggcttatggtataggcacctacaattatcagcacttctctctattgataaacaattggctgtaataccacagtagagaacgatcacaacattttgtgctggttactttttgttttatggtcatgatttcactctctctaatctgtcacttccctccattcattttgtacttctcatatttttcacttcctggttgaaaattgtagttctcttggtacatactagtattagacattcagcaacaacaactgaactgaacttctttatactttgacacagggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcattggctttcattttctacattgccaccacctacttccacctcctccctcaccccttttccctcattgcatggccaatctattgggttctccaaggttgcattcttactggcgtgtgggtgattgctcacgagtgtggtcaccatgccttcagcaagtacccatgggttgatgatgttatgggtttgaccgttcactcagcacttttagtcccttatttctcatggaaaataagccatcgccgccaccactccaacacgggttcccttgaccgtgatgaagtgtttgtcccaaaaccaaaatccaaagttgcatggtacaccaagtacctgaacaaccctctaggaagggctgcttctcttctcatcacactcacaatagggtggcctttgtatttagccttcaatgtctctggcagaccctatgatggttttgctagccactaccacccttatgctcccatatattcaaatcgtgagaggcttttgatctatgtctctgatgttgctttgttttctgtgacttacttgctctaccgtgttgcaactatgaaagggttggtttggctgctatgtgtttatggggtgccattgctcattgtgaacggttttcttgtgaccatcacatatctgcagcacacacactatgccttgcctcactatgattcatcagaatgggattggctgaggggtgctttggcaactatggacagagattatggaattctgaacaaggtgtttcaccacataactgatactcatgtggctcaccatcttttctctacaatgccacattaccatgcaacggaggcaaccaatgcaatgaagccaatattgggtgagtactaccgatttgatgacacaccattttacaaggcactgtggagagaagcaagagagtgcctctatgtggagccagatgaaggaacatccgagaagggcgtgtattggtacaggaacaagtattgatgaaccaagcaatgggccatagtgggagttatggaagttttgtcacttatcacttaattagtagaatgttataaataagtggatttgccgcgtaatgacttgtgtgcattgtgaaacagcttgtagcgatccatggctataatgtaaaaatatgtggaaagtgttctgcttataactttcttccttaaatgcacactccatagaacaa

SEQ ID NO: 25 - DNA, 1164 bp, Glycine max
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattga

SEQ ID NO: 26 - DNA, 1164 bp, Glycine max
atgggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcattggctttcattttctacattgccaccacctacttccacctcctccctcaccccttttccctcattgcatggccaatctattgggttctccaaggttgcattcttactggcgtgtgggtgattgctcacgagtgtggtcaccatgccttcagcaagtacccatgggttgatgatgttatgggtttgaccgttcactcagcacttttagtcccttatttctcatggaaaataagccatcgccgccaccactccaacacgggttcccttgaccgtgatgaagtgtttgtcccaaaaccaaaatccaaagttgcatggtacaccaagtacctgaacaaccctctaggaagggctgcttctcttctcatcacactcacaatagggtggcctttgtatttagccttcaatgtctctggcagaccctatgatggttttgctagccactaccacccttatgctcccatatattcaaatcgtgagaggcttttgatctatgtctctgatgttgctttgttttctgtgacttacttgctctaccgtgttgcaactatgaaagggttggtttggctgctatgtgtttatggggtgccattgctcattgtgaacggttttcttgtgaccatcacatatctgcagcacacacactatgccttgcctcactatgattcatcagaatgggattggctgaggggtgctttggcaactatggacagagattatggaattctgaacaaggtgtttcaccacataactgatactcatgtggctcaccatcttttctctacaatgccacattaccatgcaacggaggcaaccaatgcaatgaagccaatattgggtgagtactaccgatttgatgacacaccattttacaaggcactgtggagagaagcaagagagtgcctctatgtggagccagatgaaggaacatccgagaagggcgtgtattggtacaggaacaagtattga

SEQ ID NO: 27 - DNA, 17 bp, Artificial Sequence
  source: /note=“Description of Artificial Sequence Synthetic oligonucleotide”
tctcaagggttccaaac

SEQ ID NO: 28 - DNA, 17 bp, Artificial Sequence
  source: /note=“Description of Artificial Sequence Synthetic oligonucleotide”
ttgagttggccaacagt

SEQ ID NO: 29 - DNA, 17 bp, Artificial Sequence
  source: /note=“Description of Artificial Sequence Synthetic oligonucleotide”
tatgttgtttatgacct

SEQ ID NO: 30 - DNA, 17 bp, Artificial Sequence
  source: /note=“Description of Artificial Sequence Synthetic oligonucleotide”
tggtggcaatgtagaaa

SEQ ID NO: 31 - DNA, 17 bp, Artificial Sequence
  source: /note=“Description of Artificial Sequence Synthetic oligonucleotide”
tcctatgttgtttatga

SEQ ID NO: 32 - DNA, 17 bp, Artificial Sequence
  source: /note=“Description of Artificial Sequence Synthetic oligonucleotide”
tggcaatgtagaaaatg

SEQ ID NO: 33 - DNA, 17 bp, Artificial Sequence
  source: /note=“Description of Artificial Sequence Synthetic oligonucleotide”
ttgtttatgacctttca

SEQ ID NO: 34 - DNA, 17 bp, Artificial Sequence
  source: /note=“Description of Artificial Sequence Synthetic oligonucleotide”
taggtggtggcaatgta

SEQ ID NO: 35 - DNA, 17 bp, Artificial Sequence
  source: /note=“Description of Artificial Sequence Synthetic oligonucleotide”
tgccaccacctacttcc

SEQ ID NO: 36 - DNA, 17 bp, Artificial Sequence
  source: /note=“Description of Artificial Sequence Synthetic oligonucleotide”
tgcaatgagggaaaagg

SEQ ID NO: 37 - DNA, 366 bp, Artificial Sequence
  source: /note=“Description of Artificial Sequence Synthetic polynucleotide”
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgcttttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatga

SEQ ID NO: 38 - DNA, 360 bp, Artificial Sequence
  source: /note=“Description of Artificial Sequence Synthetic polynucleotide”
atgggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatga

SEQ ID NO: 39 - DNA, 360 bp, Artificial Sequence
  source: /note=“Description of Artificial Sequence Synthetic polynucleotide”
atgggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcatttctacattgccaccacctacttccacctcctccctcaccccttttccctcattgcatggccaatctattgggttctccaaggttgcattcttactggcgtgtgggtgattgctcacgagtgtggtcaccatgccttcagcaagtacccatgggttgatga

SEQ ID NO: 40 - DNA, 423 bp, Artificial Sequence
  source: /note=“Description of Artificial Sequence Synthetic polynucleotide”
atggttaaagacacaaagcctttagcctatgctgctaataatggataccaaaaggaagcttttgatcccagtgctcctccaccgtttaagattgcagaaatcagagttgcaataccaaaacattgctgggtcaagaatccatggagatccctcagttatgttctcagggatgtgcttgtaattgctgcattgatggctgctgcaagtcacttcaacaactggcttctctggctaatctattggcccattcaaggaacaatgttctgggctctgtttgttcttggacatgattgtggccatggaagcttttcagacagcccttttctaaatagcctggtgggacacatcttgcattcctcaattcttgtgccataccatggatggagaattagccacagaactcaccatcaatggacacattga

SEQ ID NO: 41 - DNA, 444 bp, Artificial Sequence
  source: /note=“Description of Artificial Sequence Synthetic polynucleotide”
atggttaaagacacaaagcctttagcctatgctgctaataatggataccaaaaggaagcttttgatcccagtgctcctccaccgtttaagattgcagaaatcagagttgcaataccaaaacattgctgggtcaagaatccatggagatccctcagttatgttctcagggatgtgcttgtaattgctgcattgatggctgctgcaagtcacttcaacaactggcttctctggctaatctattggcccattcaaggaacaatgttctgggctctgtttgttcttggacatgattgtggccatggaagcttttcagacagcccttttctaaatagcctggtgggacacatcttgcattcctcaattcttgtgccataccatggatggagaattagccacagaactcacctggacacattgagaaggatgaatcctgggttccattaa

SEQ ID NO: 42 - DNA, 435 bp, Artificial Sequence
  source: /note=“Description of Artificial Sequence Synthetic polynucleotide”
atggttaaagacacaaagcctttagcctatgctgccaataatggataccaacaaaagggttcttcttttgattttgatcctagcgctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaatggacacattga

SEQ ID NO: 43 - DNA, 432 bp, Artificial Sequence
  source: /note=“Description of Artificial Sequence Synthetic polynucleotide”
atggttaaagacacaaagcctttagcctatgctgccaataatggataccaacaaaagggttcttcttttgattttgatcctagcgctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaacacattga

SEQ ID NO: 44 - DNA, 468 bp, Artificial Sequence
  source: /note=“Description of Artificial Sequence Synthetic polynucleotide”
atggttcaagcacagcctctacaacatgttggtaatggtgcagggaaagaagatcaagcttattttgatccaagtgctccaccacccttcaagattgcaaatatcagagcagcaattccaaaacattgctgggagaagaacacattgagatctctgagttatgttctgagggatgtgttggtagtgactgcattggtagctgcagcaatcggcttcaatagctggttcttctggccactctattggcctgcacaaggcacaatgttttgggcactttttgttcttggacatgattgtggtcatggaagtttttcaaacagtcctttgttgaacagcattgtgggccacatcttgcactcttcaattcttgtaccataccatggatggagaattagccacaggactatggccatgttgagaaggatgaatcatgggttccgcttacagagaaagtttacaagaatctag

SEQ ID NO: 45 - DNA, 84 bp, Artificial Sequence
  source: /note=“Description of Artificial Sequence Synthetic oligonucleotide”
tttcarayagycctttkytraayagcmtkgtgggmcacatcttgcaytcytcaattcttgtrccataccatggatggtyrgttc

SEQ ID NO: 46 - DNA, 84 bp, Glycine max
tttcagacagcccttttctaaatagcctggtgggacacatcttgcattcctcaattcttgtgccataccatggatggttagttc

SEQ ID NO: 47 - DNA, 84 bp, Glycine max
tttcagatagccctttgctgaatagcctggtgggacacatcttgcattcctcaattcttgtgccataccatggatggttagttc

SEQ ID NO: 48 - DNA, 84 bp, Glycine max
tttcaaacagtcctttgttgaacagcattgtgggccacatcttgcactcttcaattcttgtaccataccatggatggtcggttc

SEQ ID NO: 49 - DNA, 70 bp, Artificial Sequence
  source: /note=“Description of Artificial Sequence Synthetic oligonucleotide”
gywkcaatwccaaaacattgctgggwsaagaaymcatkgagatcyctsagttatgttctsagggatgtgy

SEQ ID NO: 50 - DNA, 70 bp, Glycine max
gttgcaataccaaaacattgctgggtcaagaatccatggagatccctcagttatgttctcagggatgtgc

SEQ ID NO: 51 - DNA, 70 bp, Glycine max
gcttcaataccaaaacattgctgggtcaagaatccatggagatccctcagttatgttctcagggatgtgc

SEQ ID NO: 52 - DNA, 70 bp, Glycine max
gcagcaattccaaaacattgctgggagaagaacacattgagatctctgagttatgttctgagggatgtgt

SEQ ID NO: 53 - DNA, 60 bp, Artificial Sequence
  source: /note=“Description of Artificial Sequence Synthetic oligonucleotide”
tctattgsccyrywcaaggmacaatgttytgggcwctbtttgttcttggacatgattggt

SEQ ID NO: 54 - DNA, 60 bp, Glycine max
tctattggcccattcaaggaacaatgttctgggctctgtttgttcttggacatgattggt

SEQ ID NO: 55 - DNA, 60 bp, Glycine max
tctattgccccattcaaggcacaatgttctgggctctctttgttcttggacatgattggt

SEQ ID NO: 56 - DNA, 60 bp, Glycine max
tctattggcctgcacaaggcacaatgttttgggcactttttgttcttggacatgattggt

SEQ ID NO: 57 - DNA, 72 bp, Artificial Sequence
  source: /note=“Description of Artificial Sequence Synthetic oligonucleotide”
  modified_base (1)..(2): a, c, t, g, unknown or other
nnttttgcaggagaattagccacagractcaccatcaraaycatggmcayrttgagaaggatgartcmtggg

SEQ ID NO: 58 - DNA, 72 bp, Glycine max
gattttgcaggagaattagccacagaactcaccatcaaaatcatggacacattgagaaggatgaatcctggg

SEQ ID NO: 59 - DNA, 72 bp, Glycine max
gattttgcaggagaattagccacagaactcaccatcaaaaccatggacacattgagaaggatgagtcatggg

SEQ ID NO: 60 - DNA, 70 bp, Glycine max
ttttgcaggagaattagccacaggactcaccatcagaaccatggccatgttgagaaggatgaatcatggg

SEQ ID NO: 61 - DNA, 56 bp, Artificial Sequence
  source: /note=“Description of Artificial Sequence Synthetic oligonucleotide”
ctctctcaagggttccaaacacaaagccaccattcactgttggccaactcaagaaa

SEQ ID NO: 62 - DNA, 56 bp, Glycine max; identical to SEQ ID NO: 61

SEQ ID NO: 63 - DNA, 103 bp, Artificial Sequence
  source: /note=“Description of Artificial Sequence Synthetic polynucleotide”
ttcattstcctatgttgtttatgacctttcattkgcyttcattttctacattgccaccacctacttccacctcctycctcamcccttttccctcattgcatgg

SEQ ID NO: 64 - DNA, 103 bp, Glycine max
ttcattctcctatgttgtttatgacctttcatttgccttcattttctacattgccaccacctacttccacctccttcctcaacccttttccctcattgcatgg

SEQ ID NO: 65 - DNA, 103 bp, Glycine max
ttcattgtcctatgttgtttatgacctttcattggctttcattttctacattgccaccacctacttccacctcctccctcaccccttttccctcattgcatgg

(19) United States
(12) Patent Application Publication
(10) Pub. No.: US 2023/0008694 A1
(43) Pub. Date: Jan. 12, 2023

(54) TAL-EFFECTOR NUCLEASES FOR GENE EDITING

(71) Applicant: Calyxt, Inc., Roseville, MN (US)

(72) Inventor: Zachary Demorest, Roseville, MN (US)

(73) Assignee: Calyxt, Inc., Roseville, MN (US)

(21) Appl. No.: 17/780,792

(22) PCT Filed: Nov. 20, 2020
(86) PCT No.: PCT/US2020/061473

§ 371 Date: May 27, 2022

Related U.S. Application Data
(60) Provisional application No. 62/941,568, filed on Nov. 27, 2019.
(60) Provisional application No. 63/020,197, filed on May 5, 2020.

(51) Int. Cl. C12N 15/82; A01H 1/00; C12N 9/02; A01H 6/54

(52) U.S. Cl. CPC C12N 15/8213; A01H 1/104; C12N 15/8247; C12N 9/0071; A01H 6/542

(57) ABSTRACT

TALEN compositions and methods of use are disclosed, which include using multiplexing compositions to create a targeted mutation in several genes at once, such as the FAD3 A/B/C genes, compositions to create a targeted mutation in a single gene, such as a gene encoding a FAD2 protein, and combinations thereof. The compositions and methods can provide gene-edited plants, plant parts, and plant cells that have improved characteristics compared to the corresponding unaltered plants, plant parts, or plant cells. For example, soybean plants, plant parts and plant cells that are capable of producing a seed with an oil having comparatively higher levels of oleic acid and lower levels of linoleic and linolenic acid than a corresponding seed lacking the targeted mutation are also provided.

### SEQUENCE LISTING

The instant application contains a Sequence Listing which has been submitted electronically in ASCII format and is hereby incorporated by reference in its entirety. Said ASCII copy, created on Nov. 19, 2020, is named 1702_029 PCT_1_SL.txt and is 47,037 bytes in size.

### BACKGROUND

Gene editing by rare-cutting endonucleases can be used to generate deletions, insertions, and initiate homologous recombination. Transcription activator-like effector nuclease (TALEN) is a rare-cutting endonuclease that can target a specific sequence and generate a precise break in the DNA. One TALEN design process is premised on a left half-TALEN (HT) recognizing approximately 15 base pairs (bp), followed by a spacer region of approximately 15-18 bp, and a right-HT recognition sequence of approximately 15 bp (see, for example U.S. Pat. No. 8,450,471). This approach provides a high level of specificity for an intended target; however, multiple gene editing capabilities would be advantageous.

### SUMMARY

The aspects of the disclosure relate to compositions and methods for editing of genes (e.g., by introducing a mutation) using a rare cutting endonuclease, such as TALEN.

In one aspect, provided herein is a composition comprising a first nucleic acid encoding a first transcription activator-like (TAL) effector nuclease monomer capable of binding to a first half-site sequence of a first target gene, and a set of second nucleic acids. Each second nucleic acid encodes a second transcription activator-like (TAL) effector nuclease monomer capable of binding to a second half-site sequence of the first target gene or a set of second target genes. In this composition, the first half-site sequence is a conserved sequence, the first half-site sequence and each second half-site sequence are different and are separated by a spacer sequence, and the first TAL effector nuclease monomer is capable of forming a dimer with each of the second TAL effector nuclease, thus providing a set of TALEN.

In some embodiments, the dimer can cleave the target gene within a living cell when the first TAL effector nuclease monomer is bound to the first half-site sequence and the second TAL effector nuclease monomer is bound to the second half-site sequence.

In some embodiments, the first half-site sequence is a 100% conserved sequence. In some embodiments, the spacer sequence is from about 15 to about 18 nucleotides in length.

In some embodiments, the first nucleic acid comprises a FokI endonuclease domain. In some embodiments, each second nucleic acid comprises a FokI endonuclease domain.

In some embodiments, the first nucleic acid is in a vector. In some embodiments, each second nucleic acid is in a vector. In some embodiments, the first nucleic acid and the set of the second nucleic acids encoding the second TAL effector nuclease monomer are in a single vector.

In some embodiments, the first nucleic acid is a mRNA in a plasmid. In some embodiments, each second nucleic is a mRNA in a plasmid. In some embodiments, the first nucleic acid and the set of the second nucleic acids are mRNAs in a plasmid.

In some embodiments, the set of second nucleic acids comprises three or more second nucleic acids. In some embodiments, the set of second nucleic acids comprises four or more second nucleic acids.

In some embodiments, the first target gene is a gene of FAD3 family of genes of Glycine max. In some embodiments, each second target gene is a gene of FAD3 family of genes of Glycine max.

In some embodiments, the first target gene is an allele of Glycine max FAD3A gene. In some embodiments, the first target gene is an allele of Glycine max FAD3B gene. In some embodiments, first target gene is an allele of Glycine max FAD3C gene. In some embodiments, the second target gene is an allele of Glycine max FAD3A gene. In some embodiments, the second target gene is an allele of Glycine max FAD3B gene. In some embodiments, the second target gene is an allele of Glycine max FAD3C gene.

In some embodiments, the first half-site sequence is SEQ ID NO: 18. In some embodiments, wherein the composition comprises two second nucleic acids, each encoding a second transcription activator-like (TAL) effector nuclease monomer capable of binding to a second half-site sequence, wherein the second half-site sequence is SEQ ID NO: 17 or SEQ ID NO: 19.

In some embodiments, the composition further includes one or more rare cutting endonucleases targeted to an allele of the FAD2-1 family of genes of Glycine max. The one or more rare cutting endonucleases can be a TAL effector nuclease targeted to FAD2-1A or FAD2-1B. The TAL effector nuclease can include a monomer that binds to a sequence as set forth in any of SEQ ID NOS: 27-34. The TAL effector nuclease can include a pair of monomers selected from the group consisting of SEQ ID NOS: 27 and 28; 29 and 30; 31 and 32; and 33 and 34.

In some embodiments, each second target gene is a gene of FAD2 family of genes of Glycine max. In some embodiments, the first target gene is an allele of Glycine max FAD2-1A gene. In some embodiments, the first target gene is an allele of Glycine max FAD2-1B gene. In some embodiments, the second target gene is an allele of Glycine max FAD2-1A gene. In some embodiments, the second target gene is an allele of Glycine max FAD2-1B gene.

In some embodiments, the first half-site sequence is within SEQ ID NO: 25 or 26.

In some embodiments, the composition comprises two second nucleic acids, each encoding a second transcription activator-like (TAL) effector nuclease monomer capable of binding to a second half-site sequence, wherein the second half-site sequence is SEQ ID NO: 17 or SEQ ID NO: 19.

In another aspect, the present disclosure features a method of simultaneously introducing a mutation into two or more genes, including contacting a population of cells having the two or more genes with a composition according to one or more of the embodiments above.

In another aspect, the present disclosure features a plant, plant part, or plant cell obtained by the method above. The plant, plant part or plant cell can be a soybean plant, plant part or plant cell. The soybean plant parts, or plant cells can be selected from the group consisting of cotyledon cells, seeds, embryos, embryogenic calli cells, and pollen cells.

In another aspect, the present disclosure features a soybean oil composition, comprising a soybean oil produced by a soybean plant, plant part, or plant cell described in the embodiments above, wherein the soybean oil has one or more of increased oleic acid content, decreased linoleic acid content, and decreased linolenic acid content, as compared to oil produced from a corresponding soybean plant, plant part, or plant cell lacking the mutation in the two or more genes.

In another aspect, the present disclosure features a soybean plant, plant part, or plant cell having one or more mutations reducing expression of at least one of a FAD2-1A gene and a FAD2-1B gene, wherein the plant, plant part, or plant cell produces oil that has increased oleic acid content as compared to oil produced from a corresponding soybean plant, plant part, or plant cell lacking the one or more mutations, and wherein at least one mutation is induced by a rare cutting endonuclease capable of binding a nucleic acid sequence from the group set forth in SEQ ID NOs: 27-34, or a functional variant thereof.

In another aspect, the present disclosure features a method for generating a soybean plant having a mutation reducing expression of at least one of a FAD2-1A gene and a FAD2-1B gene, the method including: (a) contacting a population of soybean plant cells from a soybean plant with a functional FAD2-1A gene and FAD2-1B gene with one or more nucleic acid sequences encoding a rare cutting endonuclease capable of binding a nucleic acid sequence from the group set forth in SEQ ID NOs: 27-34, or a functional variant thereof; (b) selecting, from the population, a cell in which expression of the FAD2-1A gene or FAD2-1B gene has been reduced, and (c) regenerating the selected plant cell into a soybean plant.

In another aspect, the present disclosure features a soybean oil composition that includes a soybean oil produced by a soybean plant, plant part, or plant cell comprising one or more mutations reducing expression of at least one of a FAD2-1A gene and a FAD2-1B gene, wherein the soybean oil has one or more of increased oleic acid content, decreased linoleic acid content, and decreased linolenic acid content as compared to oil produced from a corresponding soybean plant, plant part, or plant cell lacking the one or more mutations; and wherein the one or more mutations comprise a targeted mutation induced by a rare-cutting endonuclease capable of binding a nucleic acid sequence from the group set forth in SEQ ID NOs: 27-34, or a functional variant thereof.

In another aspect, the present disclosure features a soybean plant, plant part, or plant cell comprising a first set of mutations in: one or more FAD3A alleles and one or more FAD3B alleles, one or more FAD3A alleles and one or more FAD3C alleles, one or more FAD3B alleles and one or more FAD3C alleles, or one or more FAD3A alleles, one or more FAD3B alleles, and one or more FAD3C alleles, wherein the first set of mutations is induced by expression of: a first nucleic acid encoding a first transcription activator-like (TAL) effector nuclease monomer capable of binding to a first half-site sequence of a first target gene, and a set of second nucleic acids, each second nucleic acid encoding a second transcription activator-like (TAL) effector nuclease monomer capable of binding to a second half-site sequence of the first target gene and at least one second target gene, wherein the first half-site sequence is a conserved sequence; the first half-site sequence and each second half-site sequence are different and are separated by a spacer sequence; and the first TAL effector nuclease monomer is capable of forming a dimer with each of the second TAL effector nuclease; and a mutation in: one or more FAD2-1A alleles, one or more FAD2-1B alleles, or one or more FAD2-1A alleles and one or more FAD2-1B alleles, wherein the plant, plant part, or plant cell produces oil that has decreased linolenic acid content, increased oleic acid content, and decreased linoleic acid content as compared to oil produced from a corresponding soybean plant, plant part, or plant cell lacking the mutations. The plant, plant part, or plant cell can lack a transgene. The plant part can be a seed. The mutation to the one or more FAD2-1A alleles and the one or more FAD2-1B alleles can have been induced by a rare-cutting endonuclease. The rare-cutting endonuclease can be a TAL effector nuclease. The TAL effector nuclease can bind to a sequence as set forth in any of SEQ ID NOS: 27-34. The one or more FAD3A alleles, one or more FAD3B alleles, and one or more FAD3C alleles, one or more FAD2-1A alleles and one or more FAD2-1B alleles can be mutated.

In another aspect, the present disclosure features a method for generating a soybean plant comprising a mutation reducing expression of at least two of a FAD3A gene, FAD3B gene and FAD3C gene, comprising: (a) contacting a population of soybean plant cells from a soybean plant with a functional FAD3A gene, FAD3B gene and FAD3C gene with composition comprising a first nucleic acid encoding a first transcription activator-like (TAL) effector nuclease monomer capable of binding to a first half-site sequence of a first target gene, and a set of second nucleic acids, each second nucleic acid encoding a second transcription activator-like (TAL) effector nuclease monomer capable of binding to a second half-site sequence of the first target gene and at least one second target gene, wherein the first half-site sequence is a conserved sequence; the first half-site sequence and each second half-site sequence are different and are separated by a spacer sequence; and the first TAL effector nuclease monomer is capable of forming a dimer with each of the second TAL effector nuclease, wherein the first target gene is the FAD3A gene, and the at least one second target gene is selected from the FAD3B gene and the FAD3C gene; (b) selecting, from the population, a cell in which expression of the first target gene and the at least one second target gene has been reduced, and (c) regenerating the selected plant cell into a soybean plant. The first or second monomer can be capable of binding a nucleic acid sequence from the group set forth in SEQ ID NOs: 4-19, or a functional variant thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-D show alignment of exemplary multiplex TALEN targeting FAD3 family of genes of Glycine max, according to one or more embodiments of the present disclosure: (A) alignment of GmFAD3_T01-L1, GmFAD3_T01-R1, GmFAD3_T02-L1 and GmFAD3_T02-R1 (SEQ ID NOs: 45-48, respectively, in order of appearance); (B) alignment of GmFAD3_T03-L1, GmFAD3_T03-R1, GmFAD3_T04-L1 and GmFAD3_T04-R1 (SEQ ID NOs: 49-52, respectively, in order of appearance); (C) alignment of GmFAD3_T05-L1, GmFAD3_T05-R1, GmFAD3_T06-L1 and GmFAD3_T07-L1 (SEQ ID NOs: 53-56, respectively, in order of appearance); and (D) alignment of GmFAD3_T08-L1, GmFAD3_T08-R1, GmFAD3_T09-L1, and GmFAD3_T09-R1 (SEQ ID NOs: 57-60, respectively, in order of appearance).

FIGS. 2A, B, B-1, and B-2 show alignment of exemplary TALEN targeting FAD2 family of genes of Glycine max, according to one or more embodiments of the present disclosure: (A) alignment of GmFAD2_T01-L1 and GmFAD2_T01-R1 (SEQ ID NOs: 61-62, respectively, in order of appearance) and (B) GmFAD2_T02-L1, GmFAD2_T02-R1, GmFAD2_T03-L1, GmFAD2_T03-R1, GmFAD2_T04-L1, GmFAD2_T04-R1, GmFAD2_T05-L1 and GmFAD2_T05-R1 (SEQ ID NOs: 63-65, respectively, in order of appearance). Enlarged portions of view B are presented as views B-1 and B-2 for magnification of the sequences. FIG. 2B-1 discloses residues 1-44 of SEQ ID NOs: 63-65 and FIG. 2B-2 discloses residues 45-103 of SEQ ID NOs: 63-65, all respectively, in order of appearance.

### DETAILED DESCRIPTION

Provided herein are compositions and methods for using specifically engineered rare-cutting endonuclease technology to target a single gene sequence or to simultaneously target multiple similar gene sequences, for example, genes that are part of a gene family, such as FAD3 gene family of a plant.

The rare cutting endonuclease can be a TAL effector nuclease, a meganuclease, an engineered homing endonuclease, zinc finger nuclease (ZFN) or a Clustered Regularly Interspaced Short Palindromic Repeats (CRISPR) and CRISPR-associated (Cas) system (CRISPR/Cas9). The rare-cutting endonuclease can be a natural or engineered protein having endonuclease activity directed to a nucleic acid sequence with a recognition sequence (target sequence) about 12-40 base pairs (bp) in length, or longer (e.g., 14-40, 15-36, or 16-32 by in length; see, e.g., Baker, Nature Methods 9:23-26, 2012). Typical rare-cutting endonucleases cause cleavage inside their recognition site, leaving 4 nucleotide (nt) staggered cuts with 3′OH or 5′OH overhangs. In some embodiments, the rare-cutting endonuclease is a meganuclease, such as a wild type or variant homing endonuclease (e.g., a homing endonuclease belonging to the dodecapeptide family (see, WO 2004/067736).

The rare-cutting endonuclease can be based on the RNA-guided Cas9 nuclease from the type II prokaryotic CRISPR (Clustered Regularly Interspaced Short Palindromic Repeats) adaptive immune system. This system allows for cleavage of DNA sequences that are flanked by a short sequence motif, referred as proto-spacer adjacent motif (PAM). Cleavage is achieved by engineering a specific crRNA that is complementary to the target sequence. Cas9 endonuclease will form a complex with CRISPR RNA (crRNA). A dual trans-activating crRNA (tracrRNA):crRNA structure acts as a guide RNA (gRNA) that directs the Cas9 endonuclease to the target sequence. PAM motifs in a sequence found in a FAD2-1 gene family or FAD3 gene family permit design of crRNA specific to introduce mutations or to inactivate one or more targeted genes in plant cells heterologously expressing Cas9 endonuclease and transfected with the crRNA.

In some embodiments, the rare-cutting endonuclease is a fusion protein that contains a DNA binding domain and a catalytic domain with cleavage activity. TALE-nucleases and ZFNs are examples of fusions of DNA binding domains with the catalytic domain of the endonuclease FokI. Customized TALE-nucleases are commercially available under the trade name TALEN™ (Cellectis, Paris, France). The specificity of transcription activator-like (TAL) effectors depends on an effector-variable repeat. Polymorphisms are present primarily at repeat positions 12 and 13 known as the repeat variable-diresidue (RVD). The RVDs of TAL effectors correspond to the nucleotides in their target sites in a direct, linear fashion, one RVD to one nucleotide, with some degeneracy and no apparent context dependence. This mechanism for protein-DNA recognition enables target site prediction for new target specific TAL effectors, as well as target site selection and engineering of new TAL effectors with binding specificity for the selected sites.

TAL effector DNA binding domains can be fused to other sequences, such as endonuclease sequences, resulting in chimeric endonucleases targeted to specific, selected DNA sequences, and leading to subsequent cutting of the DNA at or near the targeted sequences. Such cuts (double-stranded breaks) in DNA can induce mutations into the wild type DNA sequence via NHEJ or homologous recombination, for example. TALE-nucleases can be used to facilitate site directed mutagenesis in complex genomes, knocking out or otherwise altering gene function with great precision and high efficiency.

Methods for selecting endogenous target sequences and generating TALE-nucleases targeted to such sequences can be performed as described in the art. See, for example, PCT Publication No. WO 2011/072246, which is incorporated by reference. In some embodiments, software that specifically identifies TALE-nuclease recognition sites can be used.

Some endonucleases (e.g., FokI) function as dimers, thereby enhancing the target specificity. When two TALE-nuclease recognition sites are in proximity, the inactive monomers can come together to create a functional enzyme that cleaves the DNA. By requiring DNA binding to activate the nuclease, a highly site-specific restriction enzyme can be created.

The TALEN of the present disclosure include TALEN that are capable of targeting a plurality of genes comprising a common, e.g., conserved, sequence. The TALEN of the present disclosure comprise a common first TAL effector nuclease monomer (e.g., left half-TALEN (left-HT) or right half-TALEN or (right-HT)), and a set of second TAL effector nuclease monomers (e.g., two, three, four, or more second HT) wherein the common HT is capable of forming a dimer with any of the second HT to enable targeting of all intended sequences. As used herein, the terms “TAL effector nuclease monomer,” “half-TALEN,” and “HT” are used interchangeably. These first TAL effector nuclease monomers and sets of second TAL effector nuclease monomers can be used in combination with TALEN engineered to target a single sequence, such as a sequence of a FAD2-1 gene (e.g., at least one of FAD2-1A and FAD2-1B).

The use of the disclosed compositions and method involves alignment of the gene sequences of interest to determine regions of near conserved identity between sequences. In some embodiments, the common first TAL effector nuclease monomer (e.g., left-HT or right-HT) targets a sequence that is about 100% conserved, about 95% conserved, about 90% conserved, about 85% conserved, or about 80% conserved across all genes targeted by the compositions and methods of the disclosure.

For example, in one embodiment, a 100% conserved targeting region is selected for the left-HT binding domain (half-site sequence). For the right-HT binding domain, a unique HT for each non-conserved sequence is designed to enable targeting. In this non-limiting example, gene 1 is targeted by left-HT #1 and right-HT #1, gene 2 is targeted by left-HT #1 and right-HT #2, gene 3 is targeted by left-HT #1 and right-HT #3, and so on.

Likewise, for example, in another embodiment, a 100% conserved targeting region is selected for the right-HT binding domain. For the left-HT binding domain, a unique HT for each non-conserved sequence is designed to enable targeting. In this non-limiting example, gene 1 is targeted by left-HT #1 and right-HT #1, gene 2 is targeted by left-HT #2 and right-HT #1, gene 3 is targeted by left-HT #3 and right-HT #1, and so on.

In some embodiments, the genes targeted by the compositions and methods of the disclosure can encode the same protein or different proteins (e.g. the common HT targets a sequence that is conserved or identical in all of the genes). In some embodiments, the genes targeted by the compositions and methods of the disclosure are alleles of a gene.

In some embodiments, the targeted gene is a FAD3A, FAD3B, or FAD3C gene. In some embodiments, the targeted gene is SEQ ID NO:1-3. In some embodiments, the methods and compositions disclosed herein can target, e.g., introduce a mutation, in any two of FAD3A, FAD3B, and FAD3C genes. In some embodiments, the methods and compositions disclosed herein can target, e.g., introduce a mutation, in all three of FAD3A, FAD3B, and FAD3C genes. The one or more mutations can be present in a coding or non-coding sequence of the FAD3A, FAD3B, or FAD3C gene. Genomic sequences for native soybean genes can be found in Soybase Database (www.soybase.org). In some embodiments, the compositions and methods disclosed herein include a transcription activator-like (TAL) effector nuclease monomer capable of binding to a sequence selected from SEQ ID NOs: 4-19 or a sequence within any one of SEQ ID NOs:1-3.

In some embodiments, the methods and compositions disclosed herein can target, e.g., introduce a mutation, in any of FAD2-1A or FAD2-1B genes. In some embodiments, the targeted gene is a FAD2-1A or FAD2-1B gene. The one or more mutations can be present in a coding or non-coding sequence of the FAD2-1A or FAD2-1B gene Exemplary genomic sequences endogenous for soybean FAD2-1A and FAD2-1B genes are set forth in SEQ ID NOs: 23 and 24, respectively. In some embodiments, the targeted gene is SEQ ID NO: 23 or 24, or a functional variant thereof which has at least about 80%, such as at least about 85%, at least about 90%, at least about 95%, at least about 96%, at least about 97%, at least about 98% or at least about 99%, sequence identity with SEQ ID NO: 23 or 24. Sometimes, the targeted sequence is within the coding sequence of a FAD2-1A or FAD2-1B gene, such as the coding sequences set forth as SEQ ID NOs: 25 or 26. The target sequence can be a sequence within SEQ ID NO: 25 or 26, or a functional variant thereof which has at least about 80%, such as at least about 85%, at least about 90%, at least about 95%, at least about 96%, at least about 97%, at least about 98% or at least about 99%, sequence identity with SEQ ID NO: 25 or 26.

The percent sequence identity between any nucleic acid sequence and a sequence referenced by a sequence identification number (SEQ ID NO:) can be determined by conventional methods. In one example, a nucleic acid sequence is compared to the sequence set forth in a sequence identification number using the BLAST 2 Sequences (B12seq) program from the stand-alone version of BLASTZ containing BLASTN version 2.0.14. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide residue is presented in both sequences. The percent sequence identity is determined by dividing the number of matches either by the length of the sequence set forth in the identified sequence (e.g., SEQ ID NO: 1), or by an articulated length (e.g., 100 consecutive nucleotides from a sequence set forth in an identified sequence), followed by multiplying the resulting value by 100. The percent sequence identity value is rounded to the nearest tenth. For example, the targeted gene can have a coding sequence with at least 80%, at least 85%, at least 90%, at least 91%, at least 92%, at least 93%, at least 94%, at least 95%, at least 96%, at least 97%, at least 98%, or at least 99% sequence identity to SEQ ID NO: 20, 21, 22, 25, or 26.

In some embodiments, the compositions and methods disclosed herein include a transcription activator-like (TAL) effector nuclease monomer capable of binding to a sequence within the genomic or coding sequence of at least one of the FAD2-1A and FAD2-1B genes, For example, the TALEN monomer can be selected from one of SEQ ID NOs: 27-34, a pair of monomers selected from the group consisting of SEQ ID NOS: 27 and 28; 29 and 30; 31 and 32; 33 and 34; and 35 and 36, or functional variants of these sequences can be included. Functional variants include, for example, sequences having one or more nucleotide substitutions, deletions or insertions but retain the desired activity. Functional variants can be created by any of a number of methods available to one skilled in the art, such as by site-directed mutagenesis, induced mutation, identified as allelic variants, cleaving through use of restriction enzymes, or the like.

The compositions disclosed herein include the TALEN of the disclosure and nucleic acids encoding the TALEN of the disclosure. Thus, in some embodiments, provided herein is a composition comprising a first nucleic acid encoding a HT targeting a common sequence conserved across all genes targeted by the composition and a set of second nucleic acids encoding a plurality of second HT targeting a second sequence, wherein the HT targeting a common sequence is capable of forming a dimer with each of the second HT.

TALEN gene editing is based on the gene-specific targeting of two half-TALEN proteins containing DNA recognition domains and FokI endonuclease domains encoded in the plasmid including an appropriate promoter, terminator, and other noncoding sequences necessary for expression in the recipient cells. FokI is only active in a dimeric combination, and two half-TALEN are required to enable the endonuclease DNA cleavage necessary for TALEN gene editing.

The compositions of the disclosure can be delivered to a eukaryotic cell, a mammalian cell, a plant cell, or a prokaryotic cell. Introduction into cells can be accomplished in multiple ways known to those skilled in the art. Common methods of introduction of gene-editing TALEN include transformation with Agrobacterium spp. carrying a plasmid including TALEN gene cassettes, biolistic bombardment with plasmids or mRNA encoding TALEN sequence cassettes, and PEG-mediated transformation with plasmids or mRNA encoding TALEN sequence cassettes. In some embodiments, the compositions of the disclosure can be used to generate a set of half-TALEN proteins encoded by the compositions. The half-TALEN proteins can be isolated and subsequently introduced into a target cell, such as a plant cell, by multiple methods, for example, including PEG-mediated transfection, such as the technique described in, e.g., Luo S. et al. Non-transgenic Plant Genome Editing Using Purified Sequence-Specific Nucleases, Mol Plant. 2015 September; 8(9):1425-7.

The plant cell can be from a soybean plant. The term “soybean plant” or “plant part” is used broadly to include a soybean plant at any stage of development, or to part of soybean plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered a plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, leaves, stems, seed pods, seeds, roots, nodules, and the like. A part of a plant useful for propagation includes, for example, seeds, seed pods, cuttings, seedlings, rootstocks, and the like. “Seed” refers to any plant structure that is formed by continued differentiation of the ovule of the plant, following its normal maturation point at flower opening, irrespective of whether it is formed in the presence or absence of fertilization and irrespective of whether or not the seed structure is fertile or infertile.

The rare cutting endonuclease (e.g., TALEN) can be encoded by a nucleic acid sequence for expression in a plant cell or introduced as a protein. The nucleic acid encoding the sequence specific nuclease can be introduced to a soybean plant by Agrobacterium-mediated transformation of plant parts or plant cells (e.g., leaves, stems, petiole, internode explants, callus, or protoplasts) with T-DNA encoding the rare cutting endonuclease, biolistic transformation of plant parts or plant cells with one or more nucleic acids encoding the rare cutting endonuclease, cell-penetrating peptide-mediated transformation and/or polyethylene glycol (PEG) mediated transformation. For example, protoplasts can be isolated from surface sterilized leaves, and transformed in the presence of PEG with plasmids encoding one or more rare cutting endonucleases. Transformation efficiencies can be monitored by delivery of a detectable marker such as a YFP plasmid, which can be visualized using fluorescence microscopy or flow cytometry. After PEG-mediated transformation, protoplasts can be cultured using methods and media known to the person of ordinary skill in the art of protoplast culturing. After a suitable length of time in culture, protoplast-derived calli identified as mutants can be grown, transferred to shoot-inducing medium, and then (once roots form) transferred to soil and grown to maturity for seed production.

The rare cutting endonuclease can be delivered to the soybean plant using methods for transient expression or by using methods for stable integration into the host genome. To transiently deliver sequence-specific nucleases, transformed soybean plant parts or plant cells (e.g., using the above-described methods) can be placed on regeneration medium containing no selective agent, and soybean plants can be regenerated. Regenerated plants can be screened to identify those containing the induced mutation. To stably integrate the genome engineering reagents into the host genome, nucleic acids encoding the rare cutting endonuclease can be co-delivered with nucleic acid encoding a plant selectable marker. The selectable marker can be harbored on the same vector as the rare cutting endonuclease, or can be delivered as a separate vector. After transformation, soybean plant parts or plant cells can be placed on regeneration medium containing the appropriate selectable agent. Transformed plant cells can be regenerated into transgenic soybean plants. In some cases, the soybean plants do not include a transgene or any exogenous DNA (e.g. T-DNA). Progeny without exogenous DNA (e.g., the sequence specific nuclease sequence) can be generated by segregation. Segregation can stabilize the induced mutations as well as satisfy biosafety concerns.

In the methods above, the rare cutting endonuclease can be co-delivered to a plant cell with a plasmid encoding one or more exonuclease proteins to increase sequence specific nuclease induced mutagenesis efficiency. Such exonucleases include, without limitation, members of the TREX (three prime repair exonuclease) family of exonucleases, such as TREX2.

This disclosure provides materials and methods for using rare-cutting endonucleases (e.g., TALE-nucleases) to edit one or more genes encoding FAD2 or FADS for generating soybean plants and related products (e.g., seeds and plant parts) that are particularly suitable for providing high oleic fatty acid and/or low linoleic or linolenic fatty acid oil. The method of using the compositions of the present disclosure can include contacting a population of soybean plant cells (e.g., protoplasts) expressing one or more of a FAD2-1A gene, a FAD2-1B gene, a FAD3A gene, a FAD3B gene, and a FAD3C gene, with a rare cutting endonuclease engineered to induce a targeted mutation in the one or more genes, selecting from the population a cell with a mutation that reduces expression of the one or more genes. In addition, a method can include a step of isolating genomic DNA containing at least a portion of the one or more targeted gene loci (e.g., the FAD2-1A, FAD2-1B, FAD3A, FAD3B, or FAD3C locus) from the plant cells. In some cases, the method includes a step of culturing a plant cell containing the mutation to generate one or more plant lines. Parts of the soybean plant, such as the seeds, synthesize and accumulate fatty acids which can be identified and quantified. For example, analysis of the fatty acid composition of plant parts as described in the Examples can confirm the mutation leads to increased levels of oleic acid, decreased levels of linoleic acid, decreased levels of linolenic acid, or combinations of these characteristics in the generated plant line, as compared to a soybean plant that does not contain the mutation.

In particular embodiments, expression of the targeted gene is reduced by the induced mutation. Reducing expression of a gene in a plant, plant part or a plant cell includes inhibiting, interrupting, knocking-out, or knocking-down the gene, such that transcription of the gene and/or translation of the encoded polypeptide is reduced as compared to a corresponding control plant, plant cell, or population of plants or plant cells in which expression of the gene or polypeptide is not inhibited, interrupted, knocked-out, or knocked-down. The reduction encompasses any decrease in expression level (e.g., a decrease of 10% or more, 20% or more, 30% or more, 40% or more, 50% or more, 60% or more, 70% or more, 80% or more, 90% or more, or even 100%) as compared to the corresponding control plant, plant cell, or population of plants or plant cells. In some embodiments, reducing expression by 50% or more may be particularly useful. Expression levels can be measured using methods such as, for example, reverse transcription-polymerase chain reaction (RT-PCR), Northern blotting, dot-blot hybridization, in situ hybridization, nuclear run-on and/or nuclear run-off, RNase protection, or immunological and enzymatic methods such as ELISA, radioimmunoassay, and western blotting.

The rare cutting endonuclease can be introduced into the population of cells via a nucleic acid (e.g., a vector or a mRNA) that encodes the sequence specific nuclease, or as a protein. For example, a nucleic acid encoding a TALE nuclease targeted to a conserved nucleotide sequence present within one or more FAD2 and FAD3 genes can be used to transform soybean plant cells or plant parts (e.g., protoplasts), where they can be expressed. In some cases, TALE nuclease proteins can be introduced into soybean plant cells or plant parts (e.g., protoplasts). These cells or plant parts, or a plant cell line or plant part generated from these cells, can be analyzed to determine whether mutations have been introduced at the target site(s), using next-generation sequencing techniques (e.g., 454 pyrosequencing or Illumina sequencing). The template for sequencing can be the targeted gene amplified by PCR using primers homologous to conserved nucleotide sequences. The cells can also be analyzed for any off-target mutations.

In some embodiments, one or more nucleic acids encoding multiple TALE nucleases can be used to transform soybean plant cells or plant parts (e.g., protoplasts), where they will be expressed. These TALE nucleases can be introduced into the plant cell or part as proteins. The expressed or introduced TALE nucleases can generate double-strand breaks (DSBs) on the same chromosome resulting in the deletion of intervening sequence. These cells or plant parts, or a plant cell line or plant part generated from these cells, can be analyzed for the loss of the targeted gene. Deletion of loci containing FAD2-1 gene family or FADS gene family can be analyzed qualitative or quantitative means. For example, a first primer can be designed to be homologous to sequence upstream of the first TALE nuclease target site, and a second primer can be designed to be complementary to sequence downstream from the second TALE nuclease target site. If the targeted deletion occurred, a PCR product is obtained. Deletion of loci containing the targeted genes can also be analyzed by qPCR. A lower copy number of the targeted gene, relative to a non-modified control, can suggest the presence of the intended deletion. In a T7E1 assay, genomic DNA can be isolated from pooled calli, and sequences flanking TALE-nuclease recognition sites for the targeted gene can be PCR-amplified. Amplification products then can be denatured and re-annealed. If the re-annealed fragments form a heteroduplex, T7 endonuclease I cuts at the site of mismatch. The digested products can be visualized by gel electrophoresis to quantify mutagenesis activity of the TALE-nuclease.

Methods for contacting the population of soybean plant cells to deliver the sequence-specific nuclease can include Agrobacterium-mediated transformation of plant parts or plant cells (e.g., leaves, stems, petiole, internode explants, callus, or protoplasts) with T-DNA encoding the sequence-specific nucleases, biolistic transformation of plant parts or plant cells with one or more nucleic acids encoding the sequence-specific nucleases, and/or cell-penetrating peptide-mediated transformation of plant parts or plant cells with purified sequence-specific nucleases or nucleic acids (RNA or DNA) encoding the sequence-specific nucleases.

Polyethylene glycol (PEG) mediated transformation can be used to deliver the sequence specific nuclease. For example, protoplasts can be isolated from surface sterilized leaves, and transformed in the presence of PEG with plasmids encoding one or more sequence specific nucleases. Transformation efficiencies can be monitored by delivery of a detectable marker such as a YFP plasmid, which can be visualized using fluorescence microscopy or flow cytometry. After PEG-mediated transformation, protoplasts can be cultured using methods and media known to the person of ordinary skill in the art of protoplast culturing. After a suitable length of time in culture, protoplast-derived calli identified as mutants can be grown, transferred to shoot-inducing medium, and then (once roots form) transferred to soil and grown to maturity for seed production.

In some cases, a soybean plant includes multiple mutations directed to altering the fatty acid composition of an oil produced by the plant, plant part, or plant cell. Embodiments featuring soybean plants, plant parts or plant cells having mutations that modulate expression of the FAD2 and FAD3 proteins, wherein the plant, plant part, or plant cell produces oil that has increased oleic acid and decreased linoleic and linolenic fatty acids content as compared to oil produced from a corresponding soybean plant, plant part, or plant cell lacking the one or more mutations are within the scope of this disclosure. Targeted FAD2-1A, FAD2-1B, or FAD3A/B/C expression mutants can be implemented on any strain, species or cultivar of soybean that is of interest, without limitation. In some embodiments, the targeted gene edits or other genetic modifications can be implemented in germplasm or other plant tissue that already possesses characteristics (e.g., genetic predisposition) for higher levels of oleic acid and lower levels of linoleic or linolenic acids.

A mutant soybean line can include a mutation that provides or alters expression of proteins other than FAD2 and FAD3. The combined effect can involve the use of multiple separate nucleic acid constructs or transformation events. For example, multiple constructs as described above may be introduced into a plant cells by the same or different methods, including the introduction of such a trait by the inclusion of two transcription cassettes in a single transformation vector, the simultaneous transformation of two expression constructs, retransformation with a second expression construct, or by crossing transgenic plants via traditional plant breeding methods, so long as the resulting product is a plant having both characteristics integrated into its genome.

Conventional breeding techniques can be combined with the targeted approaches described above. In some cases, the soybean plant receiving the construct is an elite line having one or more certain agronomically important traits such as those that result in increased biomass production, increased food production, improved food quality, pest resistance, vigor, development time (time to harvest), enhanced nutrient content, novel growth patterns, flavors or colors, salt, heat, drought and cold tolerance, and the like.

The transformed soybean plants of the present disclosure can be used in a plant breeding program to create novel and useful lines and varieties. Breeding can be carried out via known procedures. DNA fingerprinting, SNP or similar technologies may be used in a marker-assisted selection (MAS) breeding program to transfer or breed mutations modulating expression of one or more of the FAD2 and FAD3 genes into other soybean plants. In some embodiments, such methods include making a cross between a soybean mutant with a second soybean plant to produce an F₁ plant, or with a species which can intercross with the mutant. The method may further include backcrossing the F1 plant to the second soybean plant and repeating the backcrossing step to generate an near isogenic line, in which the mutation is integrated into the genome of said second soybean plant; wherein the near isogenic line derived from the second plant with the integrated mutations has an altered fatty acid profile (e.g., higher levels of oleic acid, lower levels of linoleic acid, and/or lower levels of linolenic acid). Such methods can be facilitated by molecular markers or TILLING®. Thus, the mutant soybean plants of the present disclosure can be used to generate novel and useful lines and varieties.

In some embodiments, the methods described herein can be used to generate soybean varieties having oil with superior stability and performance flowing in part from the oil's fatty acid profile. Commodity soybean oil is principally composed of five fatty acids: palmitic acid (10%), stearic acid (4%), oleic acid (18%), linoleic acid (55%), and linolenic acid (13%). Soybean oil with lower linolenic acid content may increase its oxidative and frying stability. Such oil may be healthier, particularly because it does not require partial hydrogenation of the polyunsaturated fatty acids for stabilization. Partial hydrogenation produces of trans fatty acids, the consumption of which is associated with increased risk of heart disease. Soybean varieties of the present disclosure can have oil with decreased linolenic acid content, increased oleic acid content, or decreased linoleic acid content compared with a soybean variety lacking the mutations described above. For example, soybean varieties of the present disclosure with stacked mutations within the FAD2-1 and FAD3 gene families can have oil with linolenic and linoleic acid levels below 3%, and oleic acid levels over 65%.

The term “gene,” as used herein, refers to a nucleic acid sequence that includes a promoter region associated with expression of a gene product. “Gene” also encompasses intron and exon regions associated with expression of the gene product, as well as 5′ or 3′ untranslated regions associated with expression of the gene product.

An “endogenous gene” refers to a nucleic acid molecule comprising the sequence of the wild-type sequence occurring in the wild-type plant, or a sequence having a percent identity that allows it to retain the function of the encoded product, such as a sequence with at least 90% identity, and may be obtained from the plant or plant part of cell, or may be synthetically produced. Further embodiments provide the sequence has at least 91%, 92%, 93%, 94%, 95%, 96%, 97%, 98%, 99% identity.

“FAD2” refers to fatty acid desaturase 2 (FAD2) proteins, which are encoded by three FAD2 desaturase genes within the soybean genome, however, FAD2-1A (Glyma10g42470) and FAD2-1B (Glyma20g24530) are highly expressed during peak oil synthesis and are the primary genetic determinants of oleic and linoleic acid levels in soybean seeds. FAD2 catalyzes conversion of oleic to linoleic acid.

“FAD3” refers to the fatty acid desaturase 3 (FAD3) enzyme, which is produced by a family of genes consisting of FAD3A (Glyma14g37350), FAD3B (Glyma02g39230) and FAD3C (Glyma18g06950), and catalyzes the conversion of linoleic to linolenic acid.

The term “functional variant” refers to a catalytically active mutant of a recited protein or a protein domain, a nucleotide sequence encoding a mutant of a protein or protein domain performing the same function as a recited SEQ ID NO, or a nucleotide sequence variant mediating the same activity as a recited SEQ ID NO (e.g., TALEN-mediated cleavage).

As used herein, the term “about” indicates that the associated value can be modified, unless otherwise indicated, by plus or minus five percent (+/−5%) and remain within the scope of the embodiments disclosed.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise indicated, nucleic acids or oligonucleotides are written left to right in 5′ to 3′ orientation.

The words “herein”, “above”, and “below”, and words of similar import, when used in this application, refer to this application as a whole and not to any particular portions of the application, unless indicated otherwise. As used herein, the words “about” and “approximately” include minor variation around the stated value, usually within a standard margin of error, such as within 10% of the stated value.

The referenced patents, patent applications, and scientific literature referred to herein are hereby incorporated by reference in their entirety as if each individual publication, patent, or patent application were specifically and individually indicated to be incorporated by reference.

The invention will be further described in the following examples, which do not limit the scope of the invention described in the claims.

### EXAMPLES

### 1. Multiplex TALEN Design for the Fatty-Acid Desaturase 3 (FADS) Gene Family in Soybean.

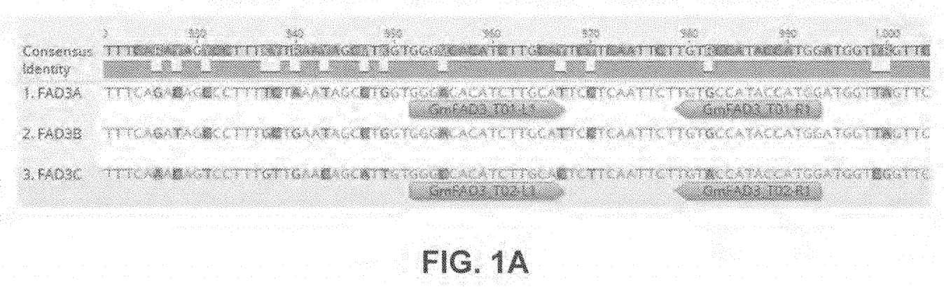

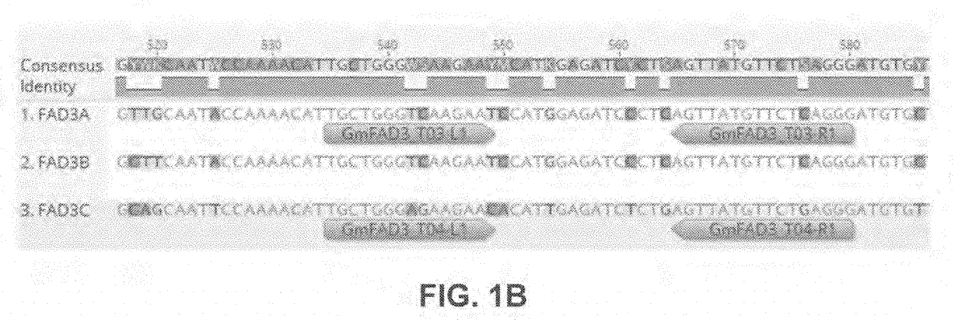

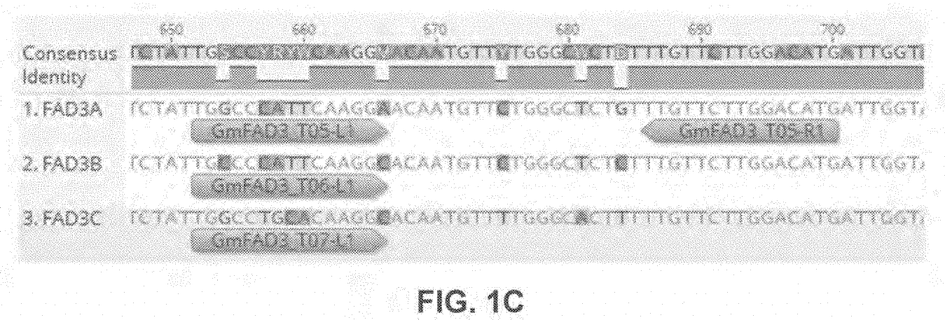

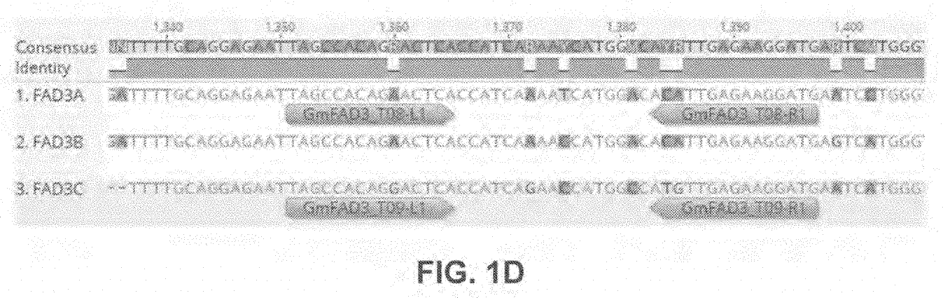

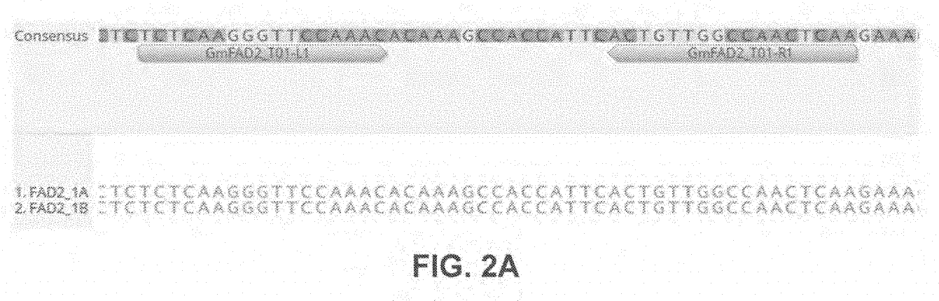

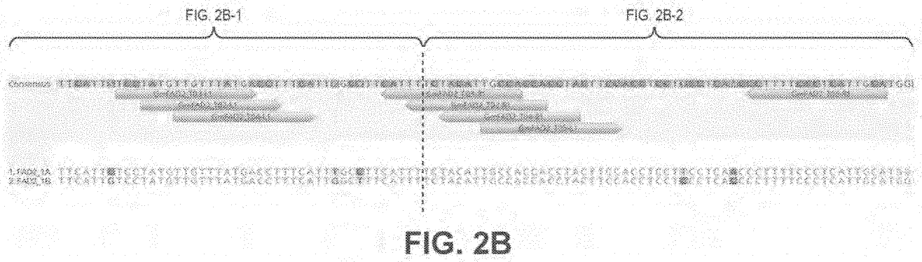

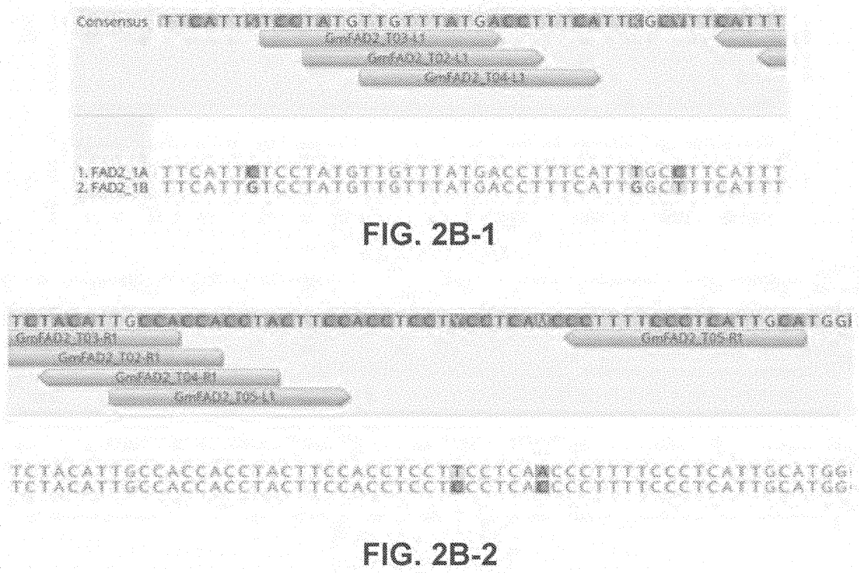

Genomic sequences for Glycine max FAD3A, FAD3B, and FAD3C (SEQ ID: 1-3) were aligned using software such as Geneious (Biomatters Ltd) and regions of homology between genes were identified. GmFAD3_T01-L1 and GmFAD3_T01-R1 (SEQ ID: 4-5) were designed with targeting specificity to FAD3A and FAD3B (SEQ ID NOs: 1-2). GmFAD3_T02-L1 and GmFAD3_T02-R1 (SEQ ID NOs: 6-7) were designed with specificity to FAD3C (SEQ ID: 3) (FIG. 1A). GmFAD3_T03-L1 and GmFAD3_T03-R1 (SEQ ID NOs: 8-9) were designed with targeting specificity to FAD3A and FAD3B (SEQ ID NOs: 1-2). GmFAD3_T04-L1 and GmFAD3_T04-R1 (SEQ ID NOs: 10-11) were designed with specificity to FAD3C (SEQ ID NOs: 3) (FIG. 1B). GmFAD3_T05-L1 (SEQ ID NO: 12) was designed with specificity to FAD3A (SEQ ID: 1), GmFAD3_T05-R1 (SEQ ID NO: 13) was designed with specificity to FAD3A, FAD3B, and FAD3C (SEQ ID NOs: 1-3), GmFAD3_T06-L1 (SEQ ID NO: 14) was designed with specificity to FAD3B (SEQ ID NO: 2), GmFAD3_T07-L1 (SEQ ID NO: 15) was designed with specificity to FAD3C (SEQ ID NO: 3) (FIG. 1C). GmFAD3_T08-L1 and GmFAD3_T08-R1 (SEQ ID NOs: 16-17) were designed with targeting specificity to FAD3A and FAD3B (SEQ ID NOs: 1-2). GmFAD3_T09-L1 and GmFAD3_T09-R1 (SEQ ID NOs: 18-19) were designed with targeting specificity to FAD3C (SEQ ID NO: 3) (FIG. 1D).

### 2. Demonstrating Nuclease Activity for HT Pairs in Soybean Protoplasts

Half-TAL-effector nucleases (half-TALEN or HT) were synthesized, cloned into plant expression vectors, and transformed into soybean protoplasts using a PEG mediated protocol to assess the activity of individual combinations of half-TALEN pairs. DNA was extracted from the protoplast population 48 hours post-transformation and PCR was used to amplify and sequence the target genes FAD3A, FAD3B, and FAD3C (SEQ ID: 1-3) using Illumina Miseq technology. Analyses were performed to determine the non-homologous end joining (NHEJ) frequency as measurement of TALEN nuclease activity. For each TALEN combination, the nuclease activity for each of the three target genes was determined. Data are summarized in TABLE 1. Using two HT-effector nucleases, the highest NHEJ frequency at FAD3A and FAD3B was achieved using combination GmFAD3_T08-L1 and GmFAD3_T08-R1 (22.87% and 14.57%). For FAD3C, the highest activity using two HT combinations was observed using GmFAD3_T09-L1 and GmFAD3_T09-R1 (11.32%).

### 3. Demonstrating Nuclease Activity for Multiplex HT in Soybean Protoplasts

A combination of three half-TAL-effector nucleases (half-TALEN or HT) with the highest activity at FAD3A, FAD3B, and FAD3C was selected and cloned into a single transformation vector for delivery into soybean protoplasts using a PEG mediated protocol. DNA was extracted from the protoplast population 48 hours post-transformation and PCR was used to amplify and sequence the target genes FAD3A, FAD3B, and FAD3C (SEQ ID NOs: 1-3) to determine NHEJ activity as above. Delivery of a combination of GmFAD3_T09-L1, GmFAD3_T08-R1, and GmFAD3_T09-R1 successfully achieved high efficiency nuclease activity at all three gene targets FAD3A (16.28%), FAD3B (14.82%), and FAD3C (21.42%). Data are summarized in Table 2.

TABLE 1
NHEJ mutagenesis frequencies for TALEN
pair combinations in protoplasts

| Left-HT | Right-HT | Target | % NHEJ |
|---|---|---|---|
| GmFAD3_T08-L1 | GmFAD3_T08-R1 | FAD3A | 22.87 |
| | | FAD3B | 14.57 |
| | | FAD3C | 3.59 |
| GmFAD3_T08-L1 | GmFAD3_T09-R1 | FAD3A | 6.56 |
| | | FAD3B | 1.80 |
| | | FAD3C | 7.60 |
| GmFAD3_T09-L1 | GmFAD3_T08-R1 | FAD3A | 12.37 |
| | | FAD3B | 11.17 |
| | | FAD3C | 5.98 |
| GmFAD3_T09-L1 | GmFAD3_T09-R1 | FAD3A | 6.46 |
| | | FAD3B | 3.61 |
| | | FAD3C | 11.32 |

TABLE 2
NHEJ mutagenesis frequencies for
Multiplex TALEN combinations in protoplasts

| Left-HT | Right-HT | Target | % NHEJ |
|---|---|---|---|
| GmFAD3_T09-L1 | GmFAD3_T08-R1 | FAD3A | 16.28 |
| | GmFAD3_T09-11 | FAD3B | 14.82 |
| | | FAD3C | 21.42 |

TABLE 3
TALEN target sequences for FAD3

| SEQ ID | Identifier | Sequence |
|---|---|---|
| 4 | GmFAD3_T01-L1 | TGGGACACATCTTGCAT |
| 5 | GmFAD3_T01-R1 | TTGTGCCATACCATGGA |
| 6 | GmFAD3_T02-L1 | TGGGCCACATCTTGCAC |
| 7 | GmFAD3_T02-R1 | TTGTACCATACCATGGA |
| 8 | GmFAD3_T03-L1 | TTGCTGGGTCAAGAATC |
| 9 | GmFAD3_T03-R1 | AGTTATGTTCTCAGGGA |
| 10 | GmFAD3_T04-L1 | TTGCTGGGAGAAGAACA |
| 11 | GmFAD3_T04-R1 | AGTTATGTTCTGAGGGA |
| 12 | GmFAD3_T05-L1 | TTGGCCCATTCAAGGAA |
| 13 | GmFAD3_T05-R1 | TTTGTTCTTGGACATGA |
| 14 | GmFAD3_T06-L1 | TTGCCCCATTCAAGGCA |
| 15 | GmFAD3_T07-L1 | TTGGCCTGCACAAGGCA |
| 16 | GmFAD3_T08-L1 | TTAGCCACAGAACTCAC |
| 17 | GmFAD3_T08-R1 | CACATTGAGAAGGATGA |
| 18 | GmFAD3_T09-L1 | TTAGCCACAGGACTCAC |
| 19 | GmFAD3_T09-R1 | CATGTTGAGAAGGATGA |

Following verification that TAL effector nuclease pairs created targeted modifications at endogenous target sites, experiments were conducted to create soybean plants with mutations in two or more FAD3A/B/C. Mutant alleles characterized from exemplary soybean plants are set forth in SEQ ID NOs: 40-44.

Seed derived from soybean lines that are homozygous mutant in two or more of FAD3A/B/C were analyzed for fatty acid composition. Briefly, individual soybean seeds are pulverized individually. DNA is prepared from a portion of the ground tissue and is analyzed to confirm the genotype of each seed. Pulverized tissue from double and triple homozygous knock out seeds is pooled. Fatty acid composition is then determined using fatty acid methyl esters (FAME) gas chromatography (Beuselinck et al., Crop Sci. 47:747-750, 2006), to assess whether seeds with various FAD3A/B/C mutations are altered in the proportion of linoleic acid, linoleic acid and oleic acid relative to wild type seed.

### 4. Sequence-Specific Nucleases to Mutagenize G. Max FAD2-1A and FAD2-1B Genes

To completely inactivate or knock-out the alleles of FAD2-1A and FAD2-1B genes in G. max, sequence-specific nucleases were designed that target the protein coding region in the vicinity of the start codon. Eight TAL effector nuclease pairs were designed to target the FAD2-1 gene family (TABLE 4). Pairs GmFAD2_T01-L1 and GmFAD2_T01-R1 (SEQ ID NOs: 27 and 28), GmFAD2_T02-L1 and GmFAD2_T02-R1 (SEQ ID NOs: 29 and 30), GmFAD2_T03-L1 and GmFAD2_T03-R1 (SEQ ID NOs: 31 and 32), and GmFAD2_T04-L1 and GmFAD2_T04-R1 (SEQ ID NOs: 33 and 34) achieved high efficiency nuclease activity at FAD2-1A and FAD2-1B gene targets (TABLE 5).

TABLE 4
TALEN target sequences for FAD2

| SEQ ID | Identifier | Sequence |
|---|---|---|
| 27 | GmFAD2_T01-L1 | TCTCAAGGGTTCCAAAC |
| 28 | GmFAD2_T01-R1 | TTGAGTTGGCCAACAGT |
| 29 | GmFAD2_T02-L1 | TATGTTGTTTATGACCT |
| 30 | GmFAD2_T02-R1 | TGGTGGCAATGTAGAAA |
| 31 | GmFAD2_T03-L1 | TCCTATGTTGTTTATGA |
| 32 | GmFAD2_T03-R1 | TGGCAATGTAGAAAATG |
| 33 | GmFAD2_T04-L1 | TTGTTTATGACCTTTCA |
| 34 | GmFAD2_T04-R1 | TAGGTGGTGGCAATGTA |
| 35 | GmFAD2_T05-L1 | TGCCACCACCTACTTCC |
| 36 | GmFAD2_T05-R1 | TGCAATGAGGGAAAAGG |

TABLE 5
NHEJ mutagenesis frequencies for TALEN pa
dr combinations in protoplasts for FAD2 target genes.

| Left-HT | Right-HT | Target | % NHEJ |
|---|---|---|---|
| GmFAD2_T01-L1 | GmFAD2_T01-R1 | FAD2-1A | 3.95 |
| | | FAD2-1B | 3.41 |
| GmFAD2_T02-L1 | GmFAD2_T02-R1 | FAD2-1A | 0.91 |
| | | FAD2-1B | 1.19 |
| GmFAD2_T03-L1 | GmFAD2_T03-R1 | FAD2-1A | 4.91 |
| | | FAD2-1B | 3.94 |
| GmFAD2_T04-L1 | GmFAD2_T04-R1 | FAD2-1A | 4.67 |
| | | FAD2-1B | 3.54 |
| GmFAD2_T05-L1 | GmFAD2_T05-R1 | FAD2-1A | 0.00 |
| | | FAD2-1B | 0.00 |

Following verification that TAL effector nuclease pairs created targeted modifications at endogenous target sites, experiments were conducted to create soybean plants with mutations in one or both of FAD2-1A and FAD2-1B. Mutant alleles characterized from exemplary soybean plants are set forth in SEQ ID NOs: 37-39.

Seed derived from soybean lines that are homozygous mutant in either FAD2-1A or FAD2-1B, or homozygous for both FAD2-1A and FAD2-1B, were analyzed for fatty acid composition. The results are shown in TABLE 6. Briefly, individual soybean seeds were pulverized individually. DNA is prepared from a portion of the ground tissue and is analyzed to confirm the genotype of each seed. Pulverized tissue from FAD2-1A homozygous, FAD2-1B homozygous, or FAD2-1A/FAD2-1B double homozygous knock out seeds is pooled. Fatty acid composition is then determined using fatty acid methyl esters (FAME) gas chromatography (as discussed above), to assess whether seeds with various FAD2-1 mutations are altered in the proportion of linoleic acid and oleic acid relative to wild type seed.

### 5. Combinations of FAD2 and FAD3 Mutations

Plants containing combinations of mutations that knock out activity of one or more FAD2 and FAD3 genes or proteins were produced, either by targeting one gene or multiple genes using TAL effector endonucleases, or by crossing plants with mutations in a FAD2 gene and/or a FAD3 gene. Plants containing a series of mutation combinations, including plants with 2, 3, 4 or 5 genes knocked out, were produced. Seed oil composition of greenhouse-grown plants was assessed by FAME. Results are shown in TABLE 6.

TABLE 6
Fatty Acid Composition

| | | | | | | | | | | | |
|---|---|---|---|---|---|---|---|---|---|---|---|
| | Myristic | Palmitic | Stearic | Oleic | Linoleic | Linolenic | Eicosanoic | Eicosenoic | Docosanoic | Erucic | Lignoceric |
| | Acid | Acid | Acid | Acid | Acid | Acid | Acid | Acid | Acid | Acid | Acid |
| SAMPLE | C14:0 | C16:0 | C18:0 | C18:1 | C18:2 | C18:3 | C20:0 | C20:1 | C22:0 | C22:1 | C24:0 |

WILD TYPE

| | | | | | | | | | | | |
|---|---|---|---|---|---|---|---|---|---|---|---|
| Soy 1 | 0.10 | 12.16 | 3.41 | 29.70 | 48.55 | 5.07 | 0.30 | 0.24 | 0.25 | 0.00 | 0.22 |
| Soy 2 | 0.10 | 12.63 | 3.19 | 20.19 | 57.07 | 5.97 | 0.25 | 0.19 | 0.25 | 0.00 | 0.15 |
| Soy 3 | 0.10 | 12.68 | 3.05 | 16.19 | 59.28 | 7.86 | 0.24 | 0.19 | 0.25 | 0.00 | 0.15 |
| Soy 4 | 0.11 | 12.83 | 3.87 | 17.17 | 57.78 | 7.24 | 0.31 | 0.19 | 0.31 | 0.00 | 0.19 |
| Soy 5 | 0.11 | 12.82 | 3.88 | 17.19 | 57.76 | 7.24 | 0.32 | 0.19 | 0.31 | 0.00 | 0.19 |
| Soy 6 | 0.00 | 12.28 | 3.90 | 22.74 | 54.28 | 6.22 | 0.32 | 0.00 | 0.26 | 0.00 | 0.00 |
| Soy 7 | 0.10 | 12.34 | 3.95 | 24.06 | 53.52 | 4.92 | 0.33 | 0.25 | 0.32 | 0.00 | 0.22 |
| Soy 8 | 0.00 | 13.43 | 3.24 | 14.82 | 58.87 | 9.06 | 0.29 | 0.00 | 0.30 | 0.00 | 0.00 |
| Soy 9 | 0.10 | 12.75 | 3.18 | 16.59 | 58.90 | 7.60 | 0.26 | 0.19 | 0.27 | 0.00 | 0.17 |
| Soy 10 | 0.10 | 12.64 | 3.74 | 16.49 | 59.03 | 7.09 | 0.28 | 0.20 | 0.27 | 0.00 | 0.15 |
| average | 0.08 | 12.66 | 3.54 | 19.51 | 56.50 | 6.83 | 0.29 | 0.16 | 0.28 | 0.00 | 0.14 |
| Low | 0.00 | 12.16 | 3.05 | 14.82 | 48.55 | 4.92 | 0.24 | 0.00 | 0.25 | 0.00 | 0.00 |
| High | 0.11 | 13.43 | 3.95 | 29.70 | 59.28 | 9.06 | 0.33 | 0.25 | 0.32 | 0.00 | 0.22 |

FIVE GENE KNOCK OUT (FAD2A/B, FAD3A/B/C)

| | | | | | | | | | | | |
|---|---|---|---|---|---|---|---|---|---|---|---|
| Soy 11 | 0.06 | 7.84 | 3.81 | 81.25 | 4.79 | 1.02 | 0.37 | 0.39 | 0.30 | 0.00 | 0.17 |
| Soy 12 | 0.00 | 8.71 | 3.79 | 79.35 | 5.98 | 1.04 | 0.38 | 0.43 | 0.33 | 0.00 | 0.00 |
| Soy 13 | 0.07 | 8.07 | 4.02 | 79.81 | 5.65 | 1.08 | 0.39 | 0.41 | 0.32 | 0.00 | 0.18 |
| Soy 14 | 0.07 | 8.77 | 3.57 | 79.56 | 5.74 | 1.14 | 0.34 | 0.38 | 0.27 | 0.00 | 0.15 |
| Soy 15 | 0.06 | 8.28 | 3.51 | 80.43 | 5.48 | 1.01 | 0.35 | 0.41 | 0.29 | 0.00 | 0.16 |
| Soy 16 | 0.00 | 9.08 | 3.30 | 78.97 | 6.49 | 1.13 | 0.33 | 0.38 | 0.33 | 0.00 | 0.00 |
| Soy 17 | 0.05 | 7.65 | 3.91 | 81.69 | 4.76 | 0.81 | 0.36 | 0.33 | 0.31 | 0.00 | 0.14 |
| Soy 18 | 0.00 | 7.69 | 3.93 | 80.96 | 5.26 | 0.92 | 0.38 | 0.40 | 0.32 | 0.00 | 0.15 |
| Soy 19 | 0.06 | 8.93 | 3.65 | 79.06 | 6.01 | 1.02 | 0.37 | 0.40 | 0.34 | 0.00 | 0.15 |
| Soy 20 | 0.00 | 9.17 | 3.43 | 78.04 | 7.09 | 1.26 | 0.36 | 0.34 | 0.31 | 0.00 | 0.00 |
| average | 0.04 | 8.42 | 3.69 | 79.91 | 5.73 | 1.04 | 0.36 | 0.39 | 0.31 | 0.00 | 0.11 |
| Low | 0.00 | 7.65 | 3.30 | 78.04 | 4.76 | 0.81 | 0.33 | 0.33 | 0.27 | 0.00 | 0.00 |
| High | 0.07 | 9.17 | 4.02 | 81.69 | 7.09 | 1.26 | 0.39 | 0.43 | 0.34 | 0.00 | 0.18 |

FOUR GENE KNOCK OUT (FAD2A/B, FAD3A/C)

| | | | | | | | | | | | |
|---|---|---|---|---|---|---|---|---|---|---|---|
| Soy 31 | 0.07 | 8.47 | 3.22 | 81.09 | 4.43 | 1.54 | 0.32 | 0.36 | 0.28 | 0.06 | 0.16 |
| Soy 32 | 0.07 | 7.99 | 3.23 | 81.53 | 2.89 | 3.11 | 0.32 | 0.36 | 0.27 | 0.07 | 0.16 |
| Soy 33 | 0.06 | 7.89 | 3.26 | 81.84 | 3.38 | 2.39 | 0.32 | 0.36 | 0.29 | 0.06 | 0.16 |
| Soy 34 | 0.07 | 8.82 | 3.13 | 79.96 | 3.48 | 3.37 | 0.31 | 0.37 | 0.27 | 0.05 | 0.16 |
| Soy 35 | 0.07 | 7.82 | 3.16 | 82.75 | 2.00 | 2.97 | 0.32 | 0.37 | 0.29 | 0.08 | 0.16 |
| Soy 36 | 0.06 | 8.45 | 3.26 | 81.14 | 3.44 | 2.38 | 0.34 | 0.39 | 0.31 | 0.06 | 0.16 |
| Soy 37 | 0.06 | 8.08 | 3.24 | 82.25 | 2.30 | 2.91 | 0.32 | 0.36 | 0.28 | 0.05 | 0.15 |
| Soy 39 | 0.06 | 8.25 | 3.23 | 81.53 | 2.52 | 3.24 | 0.31 | 0.36 | 0.28 | 0.06 | 0.14 |
| Soy 40 | 0.06 | 7.93 | 3.20 | 81.92 | 3.35 | 2.41 | 0.31 | 0.36 | 0.27 | 0.06 | 0.15 |
| average | 0.06 | 8.19 | 3.22 | 81.56 | 3.09 | 2.70 | 0.32 | 0.37 | 0.28 | 0.06 | 0.15 |
| Low | 0.06 | 7.82 | 3.13 | 79.96 | 2.00 | 1.54 | 0.31 | 0.36 | 0.27 | 0.05 | 0.14 |
| High | 0.07 | 8.82 | 3.26 | 82.75 | 4.43 | 3.37 | 0.34 | 0.39 | 0.31 | 0.08 | 0.16 |

TWO GENE KNOCK OUT (FAD2A/B)

| | | | | | | | | | | | |
|---|---|---|---|---|---|---|---|---|---|---|---|
| Soy 41 | 0.06 | 7.55 | 3.34 | 79.93 | 3.30 | 4.68 | 0.33 | 0.29 | 0.34 | 0.05 | 0.13 |
| Soy 42 | 0.07 | 7.54 | 3.49 | 78.84 | 3.21 | 5.64 | 0.35 | 0.30 | 0.35 | 0.07 | 0.15 |
| Soy 43 | 0.06 | 7.11 | 3.93 | 81.30 | 2.04 | 4.34 | 0.37 | 0.29 | 0.37 | 0.06 | 0.13 |
| Soy 44 | 0.07 | 8.22 | 3.26 | 77.19 | 4.51 | 5.47 | 0.34 | 0.33 | 0.36 | 0.09 | 0.17 |
| Soy 45 | 0.07 | 8.47 | 3.22 | 81.09 | 4.43 | 1.54 | 0.32 | 0.36 | 0.28 | 0.06 | 0.16 |
| Soy 46 | 0.07 | 7.67 | 3.28 | 78.96 | 3.74 | 5.09 | 0.32 | 0.29 | 0.35 | 0.09 | 0.15 |
| Soy 48 | 0.08 | 7.50 | 3.65 | 80.34 | 2.70 | 4.67 | 0.31 | 0.27 | 0.29 | 0.05 | 0.13 |
| Soy 50 | 0.07 | 8.02 | 3.55 | 75.61 | 4.57 | 6.89 | 0.36 | 0.29 | 0.39 | 0.09 | 0.16 |
| average | 0.07 | 7.76 | 3.47 | 79.16 | 3.56 | 4.79 | 0.34 | 0.30 | 0.34 | 0.07 | 0.15 |
| Low | 0.06 | 7.11 | 3.22 | 75.61 | 2.04 | 1.54 | 0.31 | 0.27 | 0.28 | 0.05 | 0.13 |
| High | 0.08 | 8.47 | 3.93 | 81.30 | 4.57 | 6.89 | 0.37 | 0.36 | 0.39 | 0.09 | 0.17 |

FIVE/FOUR GENE KNOCK OUTS

| | | | | | | | | | | | |
|---|---|---|---|---|---|---|---|---|---|---|---|
| Soy 71^{a} | 0.08 | 8.53 | 2.78 | 80.56 | 5.69 | 1.06 | 0.30 | 0.43 | 0.29 | 0.08 | 0.21 |
| Soy 72^{b} | 0.07 | 8.06 | 2.79 | 82.09 | 2.78 | 3.22 | 0.26 | 0.31 | 0.22 | 0.05 | 0.16 |
| Soy 73^{b} | 0.06 | 8.27 | 3.32 | 80.64 | 3.51 | 3.01 | 0.31 | 0.33 | 0.27 | 0.06 | 0.23 |
| Soy 74^{b} | 0.08 | 8.25 | 3.69 | 78.86 | 5.28 | 2.65 | 0.33 | 0.29 | 0.26 | 0.08 | 0.23 |
| Soy 75^{a} | 0.07 | 8.64 | 3.29 | 79.25 | 6.53 | 0.99 | 0.32 | 0.33 | 0.31 | 0.07 | 0.19 |
| average | 0.07 | 8.35 | 3.17 | 80.28 | 4.76 | 2.18 | 0.30 | 0.34 | 0.27 | 0.07 | 0.20 |
| Low | 0.06 | 8.06 | 2.78 | 78.86 | 2.78 | 0.99 | 0.26 | 0.29 | 0.22 | 0.05 | 0.16 |
| High | 0.08 | 8.64 | 3.69 | 82.09 | 6.53 | 3.22 | 0.33 | 0.43 | 0.31 | 0.08 | 0.23 |

^{a}5 gene KO;
^{b}4 gene KO

The fatty acid profiles show significant changes in several fatty acid levels in oil from mutant plants, relative to oil from WT plants. Linolenic, linoleic and palmitic acid levels were decreased, while the levels of eicosenoic, oleic and stearic acid were increased. In the oil of some mutants (e.g., Samples Soy 31-50 and 71-75) levels of erucic acid were increased relative to oil from WT plants.

While illustrative embodiments have been illustrated and described, it will be appreciated that various changes can be made therein without departing from the spirit and scope of the invention.

What is claimed is:

1. A composition comprising:
a first nucleic acid encoding a first transcription activator-like (TAL) effector nuclease monomer capable of binding to a first half-site sequence of a first target gene, and
a set of second nucleic acids, each second nucleic acid encoding a second TAL effector nuclease monomer capable of binding to a second half-site sequence of the first target gene or a set of second target genes,
wherein:
the first half-site sequence is a conserved sequence;
the first half-site sequence and each second half-site sequence are different and are separated by a spacer sequence; and
the first TAL effector nuclease monomer is capable of forming a dimer with each of the second TAL effector nuclease monomers.

2. The composition of claim 1, wherein the dimer can cleave the target gene within a living cell when the first TAL effector nuclease monomer is bound to the first half-site sequence and the second TAL effector nuclease monomer is bound to the second half-site sequence.

3. The composition of claim 1 or 2, wherein the first half-site sequence is a 100% conserved sequence.

4. The composition of any one of claims 1-3, wherein the spacer sequence is from about 15 to about 18 nucleotides in length.

5. The composition of any one of claims 1-4, wherein the first nucleic acid comprises a FokI endonuclease domain.

6. The composition of any one of claims 1-5, wherein each second nucleic acid comprises a FokI endonuclease domain.

7. The composition of any one of claims 1-6, wherein the first nucleic acid is in a vector.

8. The composition of any one of claims 1-7, wherein each second nucleic acid is in a vector.

9. The composition of any one of claims 1-8, wherein the first nucleic acid and the set of the second nucleic acids are in a single vector.

10. The composition of any one of claims 1-9, wherein the first nucleic acid is a mRNA in a plasmid.

11. The composition of any one of claims 1-10, wherein each second nucleic is a mRNA in a plasmid.

12. The composition of any one of claims 1-11, wherein the first nucleic acid and the set of the second nucleic acids are mRNAs in a plasmid.

13. The composition of any one of claims 1-12, wherein the set of second nucleic acids comprises three or more second nucleic acids.

14. The composition of any one of claims 1-13, wherein the set of second nucleic acids comprises four or more second nucleic acids.

15. The composition of any one of claims 1-14, wherein the first target gene is a gene of a FAD3 family of genes of Glycine max.

16. The composition of any one of claims 1-15, wherein each second target gene is a gene of a FAD3 family of genes of Glycine max.

17. The composition of any one of claims 1-16, wherein the first target gene is an allele of a Glycine max FAD3A gene.

18. The composition of any one of claims 1-16, wherein the first target gene is an allele of a Glycine max FAD3B gene.

19. The composition of any one of claims 1-16, wherein the first target gene is an allele of a Glycine max FAD3C gene.

20. The composition of any one of claims 17-19, wherein the second target gene is an allele of a Glycine max FAD3A gene.

21. The composition of any one of claims 17-19, wherein the second target gene is an allele of a Glycine max FAD3B gene.

22. The composition of any one of claims 17-19, wherein the second target gene is an allele of a Glycine max FAD3C gene.

23. The composition of any one of claims 1-22, wherein the first half-site sequence is SEQ ID NO: 18.

24. The composition of any one of claims 1-23, further comprising one or more rare cutting endonucleases targeted to an allele of a FAD2-1 family of genes of Glycine max.

25. The composition of claim 24, wherein the one or more rare cutting endonucleases is a TAL effector nuclease targeted to FAD2-1A or FAD2-1B.

26. The composition of claim 25, wherein the TAL effector nuclease comprises a monomer that binds to a sequence as set forth in any of SEQ ID NOs: 27-34.

27. The composition of claim 25, wherein the TAL effector nuclease comprises a pair of monomers that binds to a sequence selected from the group consisting of SEQ ID NOs: 27 and 28; 29 and 30; 31 and 32; and 33 and 34.

28. The composition of any one of claims 1-15, wherein each second target gene is a gene of a FAD2 family of genes of Glycine max.

29. The composition of any one of claims 1-15, wherein the first target gene is an allele of a Glycine max FAD2-1A gene.

30. The composition of any one of claims 1-15, wherein the first target gene is an allele of a Glycine max FAD2-1B gene.

31. The composition of any one of claims 28-30, wherein the second target gene is an allele of a Glycine max FAD2-1A gene.

32. The composition of any one of claims 28-30, wherein the second target gene is an allele of a Glycine max FAD2-1B gene.

33. The composition of any one of claims 1-15, wherein the first half-site sequence is within SEQ ID NO: 25 or 26.

34. The composition of any one of claims 1-33, wherein the composition comprises two second nucleic acids, each encoding a second transcription activator-like (TAL) effector nuclease monomer capable of binding to a second half-site sequence, wherein the second half-site sequence is SEQ ID NO: 17 or SEQ ID NO: 19.

35. A method of simultaneously introducing a mutation into two or more genes, comprising contacting a population of cells comprising the two or more genes with the composition of any one of claims 1-34.

36. A plant, plant part, or plant cell obtained by the method of claim 35.

37. The plant, plant part or plant cell of claim 36, wherein the plant, plant part or plant cell is a soybean plant, plant part or plant cell.

38. The plant, plant part or plant cell of claim 37, wherein the soybean plant parts, or plant cells are selected from the group consisting of cotyledon cells, seeds, embryos, embryogenic calli cells, and pollen cells.

39. A soybean oil composition, comprising a soybean oil produced by a soybean plant, plant part, or plant cell of claim 37, wherein the soybean oil has one or more of increased oleic acid content, decreased linoleic acid content, and decreased linolenic acid content, as compared to oil produced from a corresponding soybean plant, plant part, or plant cell lacking the mutation in the two or more genes.

40. A soybean plant, plant part, or plant cell comprising one or more mutations reducing expression of at least one of a FAD2-1A gene and a FAD2-1B gene, wherein the plant, plant part, or plant cell produces oil that has increased oleic acid content as compared to oil produced from a corresponding soybean plant, plant part, or plant cell lacking the one or more mutations, and wherein at least one mutation is induced by a rare cutting endonuclease capable of binding a nucleic acid sequence from the group set forth in SEQ ID NOs: 27-34, or a functional variant thereof.

41. A method for generating a soybean plant comprising a mutation reducing expression of at least one of a FAD2-1A gene and a FAD2-1B gene, comprising:
(a) contacting a population of soybean plant cells from a soybean plant with a functional FAD2-1A gene and FAD2-1B gene with one or more nucleic acid sequences encoding a rare cutting endonuclease capable of binding a nucleic acid sequence from the group set forth in SEQ ID NOs: 27-34, or a functional variant thereof;
(b) selecting, from the population, a cell in which expression of the FAD2-1A gene or FAD2-1B gene has been reduced, and
(c) regenerating the selected plant cell into a soybean plant.

42. A soybean oil composition, comprising a soybean oil produced by a soybean plant, plant part, or plant cell comprising one or more mutations reducing expression of at least one of a FAD2-1A gene and a FAD2-1B gene, wherein the soybean oil has one or more of increased oleic acid content, decreased linoleic acid content, and decreased linolenic acid content as compared to oil produced from a corresponding soybean plant, plant part, or plant cell lacking the one or more mutations; and wherein the one or more mutations comprise a targeted mutation induced by a rare-cutting endonuclease capable of binding a nucleic acid sequence from the group set forth in SEQ ID NOs: 27-34, or a functional variant thereof.

43. A soybean plant, plant part, or plant cell comprising a first set of mutations in:
one or more FAD3A alleles and one or more FAD3B alleles,
one or more FAD3A alleles and one or more FAD3C alleles,
one or more FAD3B alleles and one or more FAD3C alleles, or
one or more FAD3A alleles, one or more FAD3B alleles, and one or more FAD3C alleles,
wherein the first set of mutations is induced by expression of:
a first nucleic acid encoding a first transcription activator-like (TAL) effector nuclease monomer capable of binding to a first half-site sequence of a first target gene, and
a set of second nucleic acids, each second nucleic acid encoding a second TAL effector nuclease monomer capable of binding to a second half-site sequence of the first target gene and at least one second target gene,
wherein:
the first half-site sequence is a conserved sequence;
the first half-site sequence and each second half-site sequence are different and are separated by a spacer sequence; and
the first TAL effector nuclease monomer is capable of forming a dimer with each of the second TAL effector nuclease monomers; and
a mutation in:
one or more FAD2-1A alleles,
one or more FAD2-1B alleles, or
one or more FAD2-1A alleles and one or more FAD2-1B alleles,
wherein the plant, plant part, or plant cell produces oil that has decreased linolenic acid content, increased oleic acid content, and decreased linoleic acid content as compared to oil produced from a corresponding soybean plant, plant part, or plant cell lacking the mutations.

44. The soybean plant, plant part, or plant cell of claim 43, wherein the plant, plant part, or plant cell does not contain a transgene.

45. The soybean plant, plant part, or plant cell of claim 43, wherein the plant part is a seed.

46. The soybean plant, plant part or plant cell of claim 43, wherein the mutation to the one or more FAD2-1A alleles and the one or more FAD2-1B alleles was induced by a rare-cutting endonuclease.

47. The soybean plant, plant part or plant cell of claim 46, wherein the rare-cutting endonuclease is a TAL effector nuclease, and wherein the TAL effector nuclease binds to a sequence as set forth in any of SEQ ID NOs: 27-34.

48. The soybean plant, plant part or plant cell of claim 43, wherein one or more FAD3A alleles, one or more FAD3B alleles, and one or more FAD3C alleles, one or more FAD2-1A alleles and one or more FAD2-1B alleles are mutated.

49. A method for generating a soybean plant comprising a mutation reducing expression of at least two of a FAD3A gene, a FAD3B gene and a FAD3C gene, comprising:
(a) contacting a population of soybean plant cells from a soybean plant with a functional FAD3A gene, FAD3B gene and FAD3C gene with composition comprising a first nucleic acid encoding a first transcription activator-like (TAL) effector nuclease monomer capable of binding to a first half-site sequence of a first target gene, and a set of second nucleic acids, each second nucleic acid encoding a second TAL effector nuclease monomer capable of binding to a second half-site sequence of the first target gene and at least one second target gene, wherein the first half-site sequence is a conserved sequence; the first half-site sequence and each second half-site sequence are different and are separated by a spacer sequence; and the first TAL effector nuclease monomer is capable of forming a dimer with each of the second TAL effector nuclease monomers, wherein the first target gene is the FAD3A gene, and the at least one second target gene is selected from the FAD3B gene and the FAD3C gene;
(b) selecting, from the population, a cell in which expression of the first target gene and the at least one second target gene has been reduced, and
(c) regenerating the selected plant cell into a soybean plant.

50. The soybean plant, plant part or plant cell of claim 49, wherein the first or second monomer are capable of binding a nucleic acid sequence from the group set forth in SEQ ID NOs: 4-19, or a functional variant thereof.
